(19) United States
(12) Patent Application Publication
(10) Pub. No.: US 2012/0053222 A1
(43) Pub. Date: Mar. 1, 2012

(54) Novel Metabolic Disease Therapy

(76) Inventors: Mark Gorrell, New South Wales (AU); Sunmi Song, New South Wales (AU); Xin Wang, New South Wales (AU)

(21) Appl. No.: 13/145,906

(22) PCT Filed: Jan. 22, 2010
(86) PCT No.: PCT/AU2010/000066

§ 371 Date: Oct. 10, 2011

(30) Foreign Application Priority Data
Jan. 23, 2009 (AU) 2009900275

(51) Int. Cl. A61K 31/40; A61P 3/00; A61P 3/10

(52) U.S. Cl. 514/428

(57) ABSTRACT

The invention relates to the prevention and treatment of metabolic abnormalities characterized by abnormal glucose metabolism, including diabetes mellitus and new onset diabetes mellitus through the use of fibroblast activation protein (FAP) selective inhibitors.

### FIELD OF THE INVENTION

The invention relates to the prevention and treatment of metabolic abnormalities characterized by abnormal glucose metabolism, including diabetes mellitus and new onset diabetes mellitus.

### BACKGROUND OF THE INVENTION

Diabetes Mellitus (DM) is a syndrome of disordered metabolism and refers to the group of diseases that are associated with high blood glucose levels (hyperglycemia) due to defects in either insulin secretion (Type 1) or insulin sensitivity (Type 2).

Hyperglycemia tends to be associated with the acute forms of DM (such as diabetic ketoacidosis and hyperglycemia hyperosmolar state) and chronic forms and related complications (such as microangiopathy (including retinopathy, neuropathy, nephropathy and cardiomyopathy)) and macrovascular disease (including coronary artery disease, stroke, peripheral vascular disease, myonecrosis) and this is one reason why there has been a focus on controlling blood glucose levels in individuals having DM, in individuals having new onset DM, or in individuals at risk of these conditions.

A variety of classes of agents have been used for the control of blood glucose in patients with diabetes including:
(a) insulin sensitisers such as: glitazones (e.g. trogliazone, pioglitazone, englitazone, rosiglitazone, and the like); biguanides such as: phenformin and metformin; and protein tyrosine phosphatase 1-B inhibitors;
(b) insulin or insulin mimetics;
(c) sulfonylureas such as tolbutamide and glipizide; and
(d) a glucosidase inhibitors, examples of which include miglitol, voglibose and acarbose.

There are a number of limitations that apply to the use of some agents including side effects at doses often required for effective blood glucose control. This has limited the use of some agents in clinical practice.

There is a need for improved or alternative approaches to controlling blood glucose levels.

There is also a need for improved or alternative approaches to prevention or treatment of disease or conditions characterized by abnormal glucose metabolism.

There is also a need for improved or alternative approaches to prevention and treatment of diseases or conditions that arise as complications of DM.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

### SUMMARY OF THE INVENTION

In certain embodiments there is provided a use of a FAP specific inhibitor in one or more of the following applications:
controlling blood glucose level in an individual, especially for lowering an elevated blood glucose level in an individual resulting from food intake;
increasing insulin secretion;
decreasing glucagon secretion;
increasing p cell mass and/or insulin gene expression;
inhibiting acid secretion and gastric emptying in the stomach;
improving glucose tolerance;
decreasing food intake by increasing satiety.

In other embodiments there is provided a method of preventing or treating one or more of the following diseases or conditions:
impaired glucose tolerance;
impaired fasting glucose;
insulin resistance;
new onset diabetes mellitus;
metabolic syndrome;
diabetes mellitus;
diabetes related angiopathy and complications thereof;
hepatic steatosis;
obesity;
high fat diet induced liver damage.
   the method including providing a FAP specific inhibitor to an individual having one or more of the above diseases or conditions or, to an individual in need of treatment of one or more of the above diseases or conditions, or to an individual susceptible for one or more of the above diseases or conditions.

In certain embodiments there is provided a method of preventing the development of new onset diabetes mellitus in an individual including:
selecting an individual having a pre-diabetic state; and
administering a FAP specific inhibitor to the selected individual.

Typically the pre-diabetic state consists of one or more of metabolic syndrome, impaired glucose tolerance, impaired fasting glucose, insulin resistance and hypertension.

In other embodiments there is provided a use of a FAP specific inhibitor in the manufacture of a medicament for use in one of the above described applications.

Typically in the above described embodiments the FAP specific inhibitor is provided as the only active ingredient or active pharmaceutical principle selected for the treatment or prevention of the disease or condition.

In certain embodiments the FAP specific inhibitor may be provided in the form of a composition in which other compounds are provided as diluents, carriers, excipients or like compounds. In these forms of the invention the composition consists essentially of the FAP specific inhibitor as an active ingredient.

In other embodiments the FAP specific inhibitor may be provided in the form of a composition including other active principles or ingredients for treatment or prevention of the disease or condition.

In certain embodiments, where a FAP specific inhibitor and another active principle is to be provided for treatment or prevention of a disease or condition, the inhibitor and other active principle may be provided simultaneously, in which case they may be formulated in a composition as described above. Alternatively they may be provided from separate aliquots and administered simultaneously.

In other embodiments a FAP specific inhibitor and another active principle to be provided for treatment or prevention of a disease or condition may be provided sequentially, for example with the FAP specific inhibitor provided before the other active principle or vice versa. In these embodiments the inhibitor and the other principle are provided from separate aliquots.

In other embodiments there is provided a kit for use in one of the above described embodiments, the kit including:
a container holding a FAP specific inhibitor;
a label or package insert with instructions for use.

In certain embodiments the kit may further contain one or more active principles for treatment or prevention of the disease or condition described above.

As used herein, except where the context requires otherwise, the term “comprise” and variations of the term, such as “comprising”, “comprises” and “comprised”, are not intended to exclude further additives, components, integers or steps.

### BRIEF DESCRIPTION OF THE DRAWINGS

FIG. 1. DPIV−/− and FAP−/− mice are protected from high fat diet (HFD) induced weight gain. Percentage body weight gain in wildtype (WT), DPIV−/− and FAP−/− mice on HFD or ad libitum chow diet (chow). Percentage weight gain in DPIV−/− or FAP−/− mice on high fat diet (HFD) was significantly less than WT mice on HFD. Data are mean±SEM of 10-12 mice per group (*P<0.05 WT (HFD) versus DP4 gko (HFD) and FAP gko (HFD)). Significant differences occurred in the weeks covered by the horizontal lines.

FIG. 2. Adiposity index of wildtype (WT), FAP−/− (FAP gko) and DP4−/− (DP4 gko) mice. While WT on HFD mice exhibited significant increase in the percentage of total fat content, FAP and DP4 gko mice accumulated significantly less fat compared to WT mice fed on HFD (***P<0.0001). Data are mean±SEM of 10-12 mice per group. (ns=not significant by ANOVA).

FIG. 3. HFD increased liver weight in WT but not in FAP gko or DP4 gko mice. On chow, WT mice had heavier livers than did FAP gko and DP4 gko mice. Liver weight was measured after 12 weeks of chow or HFD from 10-12 mice per group. Mean±SEM (**P<0.01 compared to WT) (ns=not significant by ANOVA).

FIG. 4. Liver function tests after 12 weeks of HFD. (A) AST level of WT mice on HFD was significantly greater than DP4 gko mice on HFD (**P<0.01, *P<0.05 compared to WT on HFD) AST reference Interval: 20-110 U/L. (ns=not significant by ANOVA). (B) ALT level was significantly elevated in WT on HFD compared to WT on chow (*P<0.05) whereas DP4 gko and FAP gko mice on HFD had no significant increase in ALT compared to chow. ALT reference Interval: 5-55 U/L.

FIG. 5. Correlation graph of ALT and total body fat (%). (R2=0.62). (n=31, approximately equal replicates from each genotype: wildtype, FAP gko and DP4 gko mice).

FIG. 6. Oil Red O staining of lipid on a liver section from a mouse fed a HFD for 29 weeks, showing extensive lipid droplet deposition in hepatocytes. Haematoxylin counterstain of nuclei.

FIG. 7. Potential Roles of FAP inhibition in metabolism.

FIG. 8. Glucose tolerance was greater in FAP gko than wildtype mice. IPGTT of C57BL/6 and FAP gko mice after overnight fasting. The mice received a glucose load of 4 g/kg body weight at time 0 by intraperitoneal injection. Area Under Curve (AUC) data showed significantly increased glucose clearance in the FAP gko mouse strain. [Tom: THIS REFERS TO THE ADDITIONAL PANEL OF THIS FIG SENT 21ST JAN] Error bars represent SEM.

FIG. 9. Glucose tolerance was greater in FAP gko than wildtype mice. Oral Glucose Tolerance Test (OGTT) after 14 weeks of ad libitum chow (a) or HFD (b). After 5 hours of fasting, C57BL/6 and FAP gko mice received a glucose load of 2 g/kg body weight by oral gavage at time 0. Error bars represent SEM.

FIG. 10. Realtime RT-PCR analysis of lipogenic genes: SCD (a) and ACC (b). Relative mRNA expression of SCD and ACC were significantly lower in FAP gko mice on chow compared to C57BL/6 mice on chow.

FIG. 11. Tissue distribution of 1E5 binding protein in baboon tissue samples. Western blot with anti-FAP monoclonal antibody 1 E5 (Abnova, Taiwan) and a densitometry scan of the blot showed readily detectable protein in adrenal, kidney, small intestine and seminal gland.

FIG. 12. FAP gko mice were resistant to high fat diet induced weight gain.

Percentage body weight gain in wildtype (WT), and FAP gko mice on ad libitum HFD or chow diet. Mean±SEM of n=9-12 mice per group.

FIG. 13. FAP gko mice were protected against high fat diet induced adiposity. Adiposity was measured as the ratio of white fat and body mass. Results shown as mean±SEM (n=10-12 mice per group). P value of all groups <0.05 compared to WT HFD.

FIG. 14. FAP gko mice were resistant to elevated serum cholesterol level induced by HFD. Serum cholesterol level of WT, FAP gko and DPIV gko mice on ad libitum normal chow or high fat diet. Mean±SEM (n=10-12 mice per group). P value of all groups <0.05 compared to WT HFD.

FIG. 15. FAP gko mice are protected against HFD induced liver injury. ALT (a) and AST (b) of WT, FAP gko and DPIV gko mice on chow or HFD for 20 weeks Mean±SEM of n=9-12mice per group. For ALT, p value of all group <0.05 compared to WT HFD. For AST, *p value <0.05 compared to WT HFD.

FIG. 16. Correlation between ALT and total white adipose tissue mass. n=57, approximately equal replicates from each genotype: wildtype, FAP gko and DPIV gko mice

FIG. 17. FAP gko mice have improved glucose tolerance compared to WT mice. Blood glucose concentration at various time points after oral administration of glucose (given at time 0) in WT, FAP gko and DPIV gko female mice on 20 weeks HFD (a) age matched female control group on 20 weeks chow diet (b) female mice on 8 weeks HFD (c) male mice on 14 weeks HFD (d) age matched male control group on chow diet (e) n=6 mice per group mean±SEM. AUC=Area Under Curve.

FIG. 18. FAP gko mice are protected against HFD induced hyperinsulinemia. Serum insulin level of WT, FAP gko and DPIV gko mice on normal chow or 8 weeks of HFD diet. Mean±SEM, n=6 mice per group. All groups have p value <0.05 compared to WT HFD.

FIG. 19. FAP gko mice had greater insulin sensitivity compared to WT mice after HFD treatment. Blood glucose concentration measured at various times before and after insulin administration (given at time 0) to WT, DPIV gko and FAP gko mice on 20 weeks HFD (a) and control group on chow diet (b) n=6 mice per group, Mean±SEM. HOMA IR is a standard Insulin Resistance calculation.

FIG. 20. FAP gko mice showed reduced food intake compared to WT. Average food intake/day of WT, FAP gko and DPIV gko mice on HFD measured over 4 days of light and dark cycles. Mean±SEM of n=8 (2 cages per group, each cage containing 3 mice). *p<0.05 compared to WT, # p<0.05 compared to FAP gko.

FIG. 21. Intrahepatic Foxo1 mRNA level after 20 weeks. Mean±SEM of n=9-12 mice per group. # p<0.05 compared to wt HFD; ♦p<0.05 compared to FAP gko HFD.

Mann Whitney test:
WT Chow vs WT HFD p=0.1135
WT HFD vs FAP gko HFD p=0.2545
WT HFD vs DPIV gko HFD p=0.2242
WT HFD vs FAP gko chow p=0.0229
WT HFD vs DPIV gko chow p=0.0172

FIG. 22. Intrahepatic Glucokinase mRNA level. Mean±SEM of n=4-6 mice per group. All groups had p value <0.05 compared to WT HFD. p<0.05 compared to WT Chow; # p<0.05 compared to WT HFD.

FIG. 23. Intrahepatic CD36 mRNA . mice on chow and HFD for 20 weeks. Mean±SEM of n=4-6 mice per group. All groups had p<0.05 compared to WT HFD.* p<0.05 compared to WT chow; # p<0.05 compared to WT HFD. ♦p<0.05 compared to FAP gko HFD

FIG. 24. Intrahepatic IRS-2 mRNA level in WT, FAP gko and DPIV gko mice on chow and HFD for 20 weeks. Mean±SEM of n=9-12 mice per group. *p value <0.05 compared to WT chow. ▪ p value <0.05 compared to DPIV gko chow.

FIG. 25. Intrahepatic G6PC mRNA level in WT, FAP gko and DPIV gko mice on chow and HFD Mean±SEM of n=9-12 mice per group. *p<0.05 compared to WT chow; # p<0.05 compared to WT HFD; ▪ p<0.05 compared to DPIV gko chow; ♦ p<0.05 compared to FAP gko chow .

FAP Chow vs FAP HFD p=0.026

FIG. 26. Intrahepatic ChREBP mRNA level in WT, FAP gko and DPIV gko mice on chow and HFD. Mean±SEM of n=9-12 mice per group. # p value <0.05 compared to wt HFD ; ♦p p value <0.05 compared to FAP gko HFD
WT Chow vs WT HFD p=0.1011
WT HFD vs FAP gko Chow p=0.0078
WT HFD vs DPIV gko Chow p=0.0155

FIG. 27. Intrahepatic Fabp5 mRNA level in WT, FAP gko and DPIV gko mice on chow and HFD. Mean±SEM of n=9-12 mice per group. *p<0.05 compared to WT Chow; # p<0.05 compared to WT HFD.

FIG. 28. Intrahepatic DPIV mRNA level in WT, FAP gko and DPIV gko mice on chow and HFD. Mean±SEM of n=9-12 mice per group.

FIG. 29. Correlation between DPIV and G6PC and ChREBP mRNA levels in liver. (a) DPIV and G6PC (b) DPIV and ChREBP in WT and FAP gko mice (pooled data from the two genotypes). Data were calculated as number of molecules relative to 18S. Individual replicates are plotted (n=43, approximately equal replicates from each genotype: wildtype and FAP gko).

FIG. 30. Circulating adiponectin levels (a); adiponectin concentration per gram body fat (b) in WT, FAP gko and DPIV gko mice on HFD or chow diet for 20 weeks. Results are shown as mean±SEM (n=4 to 6 mice per group). *p<0.05 compared to WT chow. # p<0.05 compared to DPIV gko chow. Star, p value <0.05 compared to FAP gko chow. Y axis units: ng/ml/g).

FIG. 31. Intrahepatic PPARα (a), SREBPI-c (b), CPTI (c), AOX (d), and HNF4α mRNA level in WT, FAP gko and DPIV gko mice on chow and HFD for 20 weeks. Mean±SEM of n=9 to 12 mice per group. *P value<0.05 compared to FAP gko chow.

FIG. 32. Plasma active GLP-1 levels at fasting (a) and 15 mins post oral glucose challenge (b) in WT, FAP gko and DPIV gko mice fed on HFD or normal chow diet. Mean±SEM of n=4 to 6 mice per group. *p<0.05 compared to DPIV gko chow.

FIG. 33. A and B. FAP gko mice exhibited improved glucose tolerance from one week of CCI4 treatment. All glucose responses in glucose tolerance tests (GTT) are given as area under curve (AUC) (A). GTT data at 3 weeks of CCl4 (B). Mean±SEM of n=8 mice per group

FIG. 34. FAP gko mice have improved glucose tolerance with severity of CCI₄ treatment. GTT data before (b) and after CCI4 treatment in WT, DPIV gko and FAP gko (a) mice. Mean±SEM of n=8 mice per group.

### APPENDICES

Appendix 1. Percentage weight gain of Mice fed on HFD from Specialty Feeds, Research Diet, Test HFD or normal chow diet.

Appendix 2. Non-fasting plasma glucose levels.

Appendix 3. Liver to body weight ratio of WT, FAP and DP4 gko mice on normal chow diet or HFD.

Appendix 4. Total plasma protein of WT, FAP and DP4 gko mice on normal chow diet or HFD.

Appendix 5. Spleen to body weight ratio of WT, FAP and DP4 gko mice on normal chow diet or HFD.

Appendix 6. Plasma bilirubin levels of WT, FAP and DP4 gko mice on normal chow diet or HFD.

Appendix 7. Plasma albumin levels of WT, FAP and DP4 gko mice on normal chow diet or HFD.

### DETAILED DESCRIPTION OF THE EMBODIMENTS

Reference will now be made in detail to certain embodiments of the invention, examples of which are illustrated in the accompanying structures and formulas. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

### I Definitions

Unless stated otherwise, the following terms and phrases as used herein are intended to have the following meanings:

The words “treat” or “treatment” refer to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

The words “prevent” and “prevention” refer to therapeutic prophylactic or preventative measures for protecting or precluding an individual not having a given condition from progressing to that condition. Individuals in which prevention is required include those who are prone or predisposed to a condition.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. In certain embodiments a therapeutically effective amount may achieve one or more of lowering blood glucose level, increasing insulin secretion, decreasing glucagon secretion, decreasing insulin resistance and increasing insulin sensitivity.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable” indicates that the substance or composition must be compatible chemically and/or toxicologically, with the other ingredients comprising a formulation, and/or the mammal being treated therewith.

“Impaired glucose tolerance” is defined here on the basis of American Diabetes Association criteria. Impaired glucose tolerance is two-hour 75-g oral glucose tolerance test values of 140 to 199 mg per dL (7.8 to 11.0 mmol/l).

“Impaired fasting glucose” is defined here on the basis of American Diabetes Association criteria. Impaired fasting glucose is defined as fasting plasma glucose values of 100 to 125 mg per dL (5.6 to 6.9 mmol/l).

“Diabetes Mellitus” generally refers to fasting plasma glucose values of ≧126 mg/dL (≧7.0 mmol/l).

“Insulin resistance” is defined here as a fasting blood insulin level greater than 20 mcU/mL.

“New onset diabetes” (usually defined on the basis of a fasting blood glucose concentration of 7.0 mmol/l or more) in an individual.

A “pre-diabetic state” is a condition often preceding new onset diabetes and may be characterised by metabolic syndrome, impaired glucose tolerance, impaired fasting glucose or insulin resistance.

“Metabolic syndrome” or “syndrome X” is defined here on the basis of NCEP ATP III criteria, which are the presence of three or more of the following factors: 1) increased waist circumference (>102 cm [>40 in] for men, >88 cm [>35 in] for women); 2) elevated triglycerides (>150 mg/dl); 3) low HDL cholesterol (<40 mg/dl in men, <50 mg/dl in women); 4) non-optimal blood pressure (>130 mmHg systolic or mmHg diastolic); and 5) impaired fasting glucose (>110 mg/dl).

Generally “hyperglycemia” is a fasting blood glucose concentration of 7.0 mmol/l or greater.

“Hepatic steatosis” refers to a process describing the abnormal retention of lipids within a hepatocyle. Steatosis may result from obesity, insulin resistance, alcoholism or viral infection.

The term fibroblast activation protein alpha (UniProtKB/Swiss-Prot Q12884), herein abbreviated “FAP”, refers to a serine protease that possesses dipeptidyl-peptidase activity specific for N-terminal Xaa-Pro sequences. In addition to the dipeptidyl peptidase activity, FAP also possesses collagenolytic activity capable of degrading gelatin and type I collagen and endopeptidase activity. FAP is a type II transmembrane serine protease which is expressed as a homodimer. The 95-kDa protein exhibits 48% amino acid identity with DPIV and displays structural similarity to other members of the dipeptidyl peptidase family including DP8 and DP9. Unlike DPIV, FAP has also been reported to possess endopeptidase activity FAP overexpression has been shown to potentiate tumour growth, and this potentiation may be dependent upon its enzymatic activity. FAP is expressed on stromal fibroblasts in more than 90% of carcinomas including breast, colon, ovarian, bladder and pancreas as determined by immunohistochemistry, and this is where the focus of research into FAP has been to date i.e. as a very specific target for potential anti-tumour agents and as a biomarker of cancer. FAP substrates include collagen and alpha2 antiplasmin (Aggarwal 2008; Lee 2006; refs 17, 18). Natural substrates for FAP relevant to metabolism have not been identified.

The terms “FAP specific inhibitor” or “FAP selective inhibitor” refer to a compound that inhibits or reduces one or more of the following enzymatic activities of FAP:
dipeptidyl peptidase activity
endopeptidase activity
collagenase activity.

In certain embodiments a FAP specific inhibitor inhibits dipeptidyl peptidase activity only.

In certain embodiments, the FAP specific inhibitor does not substantially inhibit the activity of another dipeptidyl peptidase, especially DPIV (otherwise known as CD26). By “does not substantially inhibit” is generally meant that particularly preferred FAP specific inhibitors are compounds that are competitive inhibitors with IC₅₀ of <IμM for FAP and IC₅₀>IμM for DPIV, DP8 and DP9. Assays for determining competitive inhibition are described further herein and include a fluorogenic assay utilising dipeptide-AFC substrates. For example, inhibitory activity may be measured using H-Ala-Pro-AFC as a substrate using a method described in WO 9515309.

In other embodiments, the FAP specific inhibitor inhibits FAP activity and may also inhibit the activity of another dipeptidyl peptidase especially DP8 and/or 9; or PEP (prolylendopeptidase). In these embodiments the FAP specific inhibitor does not inhibit DPIV activity.

### II FAP Specific Inhibitors

Examples of FAP specific inhibitors are shown below:

(2S)-I-((2 S)-2 -(2-Methoxybenzoylamino)-3-methylpentanoyl) pyrrolidine-2-carbonitrile;

(2S)-I-((2 S)-2 -(Ethylcarbamoylamino)-3-methylpentanoyl) pyrrolidine-2-carbonitrile;

(2S)-I-((2 S)-2 -(3-Methoxybenzoylamino)-3-methylpentanoyl) pyrrolidine-2-carbonitrile;

(2S)-I-((2 S)-2 -(4-Methoxybenzoylamino)-3-methylpentanoyl) pyrrolidine-2-carbonitrile;

(2S)-I-((2 S)-2 -(Pivaloylamino)-3-methylpentanoyl) pyrrolidine-2-carbonitrile;

(2S)-I-((2 S)-I-(3-Methoxybenzoyl)pyrrolidine-2-carbonyl) pyrrolidine-2-carbonitrile;

(2S)-I-((2 S)-I-(Isopropylcarbamoyl)pyrrolidine-2-j carbonyl) pyrrolidine-2-carbonitrile;

(2S)-I-((2 S) -2-(2-Methoxybenzoylamino)-3,3-dimethylbutanoyl) pyrrolidine-2-carbonitrile;

(2S)-I-((2 S)-I-(3-Chlorobenzoyl)-pyrrolidine-2-carbonyl) pyrrolidine-2-carbonitrile;

(2S)-I-((2 S)-2-(3-Methoxybenzoylamino)-4-methylpentanoyl) pyrrolidine-2-carbonitrile;

(2S)-I-((2 S)-2-(2 -Phenylethylthiocarbamoylamino)-3-methylpentanoyl) pyrrolidine-2-carbonitrile;

(2S)-I-((2 S)-I-(Propylthiocarbamoyl)pyrrolidine-2-carbonyl) pyrrolidine-2-carbonitrile;

(2S)-I-(2-(N-(2-Methoxybenzoyl)-N-(2-methylpropyl) amino) acetyl) pyrrolidine-2-carbonitrile;

(2S)-I-((2 S)-2-(2-Methoxybenzoylamino)-5-amino-pentanoyl) pyrrolidine-2-carbonitrile;

(2S)-I-((2 S)-2-(2-Methoxybenzoylamino)-6-amino-hexanoyl) pyrrolidine-2-carbonitrile;

(4R)-I-((2 S)-2-(3-Methoxybenzoylamino)-3-methylpentanoyl) thiazolidine-4-carbonitrile;

(2S)-I-((2 S)-2-(2-Methoxybenzoylamino)-3-methylbutanoyl)-4, 4-difluoropyrrolidine-2-carbonitrile;

(2S)-I-((2 S)-2 -(Ethylcarbamoylamino)-3-methylbutanoyl)-4, 4-difluoropyrrolidine-2-carbonitrile;

(2S)-I-((2 S)-I-(Ethylcarbamoyl)pyrrolidine-2-carbonyl)-4, 4-difluoropyrrolidine-2-carbonitrile;

(2S)-I-((2 S)-2-(2-Methoxybenzoylamino)-3-methylpentanoyl)-3, 4-dehydropyrrolidine-2-carbonitrile;

(2S)-I-((2 S)-2-(2-Methoxybenzoylamino)-3-methylpentanoyl) piperidine-2-carbonitrile;

(2S)-I-((2 S)-2-(2-Methoxybenzoylamino)-3-methylpentanoyl) azetidine-2-carbonitrile;

(4R)-I-((2 S)-2-(3-Methoxybenzoylamino)-3-methylpentanoyl) thiazolidine-4-carbonitrile-S-oxide;

(4R)-I-((2 S)-2-(3-Methoxybenzoylamino)-3-methylpentanoyl) thiazolidine-4-carbonitrile-S, S-dioxide;

I-((2 S)-2-(2-Methoxybenzoylamino)-3-methylpentanoyl) pyrrolidine; I-((2 S)-2-(2-Methoxybenzoylamino)-3-methylpentanoyl)-

3, 3-difluoropyrrolidide; I-(2 S)-I-(3-Methoxybenzoyl)pyrrolidine-2-carbonyl)-3-thiazolidide; I-((2 S)-I-(Isopropylcarbamoyl) pyrrolidine-2-carbonyl) pyrrolidide;

(2R)-I-((2 S)-2-(2-Methoxybenzoylamino)-3-methylbutanoyl) pyrrolidine-2-boronic acid;

(2R)-I-((2 S)-2-(Ethylcarbamoylamino)-3-methylbutanoyl) pyrrolidine-2-boronic acid;

(2S)-2-Formyl-I-((2 S)-2-(2-methoxybenzoylamino)-3-methylpentanoyl) pyrrolidide;

(2S) -2-Acetyl-I-((2 S)-2-(2-methoxybenzoylamino)-3-methylpentanoyl) pyrrolidide;

(2S)-2-Propionyl-I-((2 S)-2-(2-methoxybenzoylamino)-3-methylpentanoyl) pyrrolidide;

(2S)-1-(2-(1-Napthoylamino) acetyl) pyrrolidine-2-carbonitrile;

(2S)-I-((2 S)-2-(4-Methylbenzoyl)octahydroindole-2-carbonyl) pyrrolidine-2-carbonitrile;

(2S)-I-((2 S)-2-(4-Methoxybenzoylamino)-2-cyclohexylacetyl) pyrrolidine-2-carbonitrile;

(2S)-I-((2 S)-I-Benzoyl piperidine-2-carbonyl) pyrrolidine-2-carbonitrile;

(2S)-I-((2 S)-2-(3-Methoxybenzoylamino)-3-methylbutanoyl) pyrrolidine-2-carbonitrile;

(2S)-I-((2 S)-2-(Pivaloylamino)-3-phenylpropanoyl) pyrrolidine-2-carbonitrile;

(2S)-I-((2 S)-2-(N-Cyclohexanoyl-N-methylamino)-3-phenylpropanoyl) pyrrolidine-2-carbonitrile;

(2S)-I-((2 S)-2-(2-Methoxybenzoylamino)-3 -(methyloxycarbonyl) propanoyl) pyrrolidine-2-carbonitrile;

(2S)-I-((2 S)-2-(4-Chlorobenzoylamino)-4-(benzyloxycarbonyl) butanoyl) pyrrolidine-2-carbonitrile;

(2S)-I-((2 S)-2-(4-Methoxybenzoylamino)-5-(benzyloxycarbonylamino) pentanoyl) pyrrolidine-2-carbonitrile;

(2S)-I-((2 S)-2-(3-Methoxybenzoylamino)-6-(benzyloxycarbonylamino) hexanoyl) pyrrolidine-2-carbonitrile;

(2S)-I-((2 S)-2-(Benzoylamino)-3-benzyloxypropanoyl) pyrrolidine-2-carbonitrile;

(2S)-I-((2 S)-2-(Cyclohexanoylamino)-2-phenylacetyl) pyrrolidine-2-carbonitrile;

(2S)-I-((2 S)-2-(2-Methoxybenzoylamino)-4-(benzyloxycarbonylamino) butanoyl) pyrrolidine-2-carbonitrile;

(2S)-I-((2 S)-2-(2-Methoxybenzoylamino)-3-(benzyloxycarbonylamino) propanoyl) pyrrolidine-2-carbonitrile;

(2S)-I-((2 S)-2-(N-Cyclohexanoyl-N-methylamino)-propanoyl) pyrrolidine-2-carbonitrile;

(2S)-I-((2 S)-2-(2-Methoxybenzoylamino)-5-N-(acetylamino)-3, 3-dimethyl-4-thiopentanoyl) pyrrolidine-2-carbonitrile;

(2S)-I-((2 S)-2-(N-(2-Methoxybenzoyl)-N-methylamino)-3 ▪ methylpentanoyl) pyrrolidine-2-carbonitrile;

(2S)-I-(2-(N-Benzyl-N-(2-methoxybenzoyl) amino) acetyl) pyrrolidine-2-carbonitrile;

(2S)-I-(2-(2-Methoxybenzoylamino)-2-methylpropanoyl) pyrrolidine-2-carbonitrile;

(2S)-I-((2 S)-2-(Ethylcarbamoylamino)-3-methylbutanoyl) pyrrolidine-2-carbonitrile;

(2S)-I-((2 S)-2-(Isopropylcarbamoylamino) propanoyl)-pyrrolidine-2-carbonitrile;

(2S)-I-((2 S)-2-(Ethylcarbamoyl)octahydroindole-2-carbonyl) pyrrolidine-2-carbonitrile;

(2S)-I-((2 S)-2-(N-Ethylcarbamoyl-N-methylamino)-4-methylpentanoyl) pyrrolidine-2-carbonitrile;

(2S)-I-((2 S)-2-(Benzylcarbamoylamino)-3, 3-dimethylbutanoyl) pyrrolidine-2-carbonitrile;

(2S)-I-((2 S)-2-(Isopropylcarbamoylamino)-3-phenylpropanoyl) pyrrolidine-2-carbonitrile;

(2S)-I-((2 S)-2-(Cyclohexylcarbamoylamino)-4-methylpentanoyl) pyrrolidine-2-carbonitrile;

(2S)-I-((2 S)-2 -(Ethylcarbamoylamino)-3-methylpentanoyl)-3, 4-dehydropyrrolidine-2-carbonitrile;

(2S)-2-Formyl-I-((2 S)-2-(2-methoxybenzoylamino)-3-methylbutanoyl) pyrrolidine;

(2S)-2-Formyl-I-((2 S)-I-(3-methoxybenzoyl) pyrrolidine-2-carbonyl) pyrrolidine;

(2S)-2-Formyl-I-((2 S)-I-(ethylcarbamoyl) octahydroindole-2-carbonyl) pyrrolidine;

(2S)-2-Formyl -I-((2 S)-2-(2-methoxybenzoyl)-octahydroindole-2-carbonyl) pyrrolidine; and

(2S)-2-Formyl-I-((2 S) -2-(2-methoxybenzoylamino) -3-phenylpropanoyl) pyrrolidine.

In one embodiment the FAP specific inhibitor is selected from the group represented by Formula A:

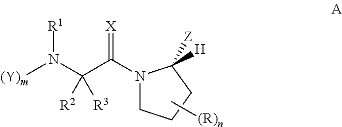

or a pharmaceutically acceptable salt thereof, wherein, independently for each occurrence:
X represents O, S, or NR;
Y represents H, naturally occurring L-amino acid residue, naturally occurring D-amino acid residue, or N-terminal protecting group;
Z represents —CO₂R′, —SO₃H, —SO₂NH₂, —B(OH)₂, —PO₃H₂, or 5-tetrazolyl;
R represents independently for each occurrence H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, or arylsulfonyl;
R¹ represents H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, or arylsulfonyl;
R² represents H, a side chain of a naturally occurring amino acid, or a side chain of a non-naturally occurring amino acid;
R³ represents H, a side chain of a naturally occurring amino acid, or a side chain of a non-naturally occurring amino acid;
R¹ and R² may be taken together to form an 3-8 member ring that may be optionally substituted;
R′ represents, independently for each occurrence, H, alkyl, alkenyl, alkynyl, aryl, heteroaryl, aralkyl, or heteroaralkyl;
m is an integer in the range 1 to about 10; and
n is an integer in the range 0 to 6.

In one embodiment, X is O.

In one embodiment, Z represents —CO₂R′or —B(OH)₂.

In one embodiment, Z represents —CO₂R′, and R′ represents H.

In one embodiment, Z represents —B(OH)₂.

In one embodiment, R¹ and R² are taken together to form a five-membered ring, giving the amino acid residue proline.

In one embodiment, X is O; Z is —B(OH)₂; R¹ is H; R² is the side chain of the amino acid residue tryptophan; R³ is H; m is 1; Y is H or an N-terminal protecting group; and n is 0.

In one embodiment, X is O; Z is —B(OH)₂; R¹ is H; R² is the side chain of the amino acid residue tryptophan; R³ is H; m is 1; Y is H; and n is 0.

In one embodiment, X is O; Z is —B(OH)₂; R¹ is H; R² is the side chain of the amino acid residue tryptophan; R³ is H; m is 1; Y is an N-terminal protecting group; and n is 0.

In one embodiment, X is O; Z is —B(OH)₂; R¹ is H; R² is the side chain of the amino acid residue tryptophan; R³ is H; m is 1; Y is Ac; and n is 0.

In one embodiment, X is S.

In one embodiment, Z represents —CO₂R′ or —B(OH)₂.

In one embodiment, Z represents —CO₂R′, and R′ represents H.

In one embodiment, Z represents —B(OH)₂.

In one embodiment, R¹ and R² are taken together to form a five-membered ring, giving the amino acid residue proline.

In one embodiment, X is S; Z is —B(OH)₂; R¹ is H; R² is the side chain of the amino acid residue tryptophan; R³ is H; m is 1; Y is H or an N-terminal protecting group; and n is 0.

In one embodiment, X is S; Z is —B(OH)₂; a^{l} is H; R² is the side chain of the amino acid residue tryptophan; R³ is H; m is 1; Y is H; and n is O.

In one embodiment, X is S; Z is —B(OH)₂; R¹ is H; R² is the side chain of the amino acid residue tryptophan; R³ is H; m is 1; Y is an N-terminal protecting group; and n is 0.

In one embodiment, X is S; Z is —B(OH)₂; R¹ is H; R² is the side chain of the amino acid residue tryptophan; R³ is H; m is 1; Y is Ac; and n is 0.

In further embodiments, the FAP specific inhibitor is selected from the group represented by Formula B:

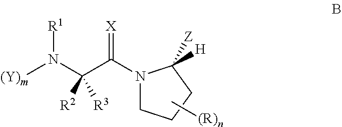

or a pharmaceutically acceptable salt thereof, wherein, independently for each occurrence:
X represents O, S, or NR;
Y represents H, naturally occurring L-amino acid residue, naturally occurring D-amino acid residue, or N-terminal protecting group;
Z represents —CO₂R′, —SO₃H, —SO₂NH₂, —B(OH)₂, 1'PO₃H₂, or 5-tetrazolyl;
R represents independently for each occurrence H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, or arylsulfonyl;
R¹ represents H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy) carbonyl, (arylalkyloxy)carbonyl, (alkylamino) carbonyl, (arylamino)carbonyl, (arylalkylamino)carbonyl, alkylsulfonyl, or arylsulfonyl;
R² represents H or a side chain of a naturally occurring amino acid;
R³ represents H or a side chain of a non-naturally occurring amino acid;
R¹ and R² may be taken together to form an 3-8 member ring that may be optionally substituted;
R′ represents, independently for each occurrence, H, alkyl, alkenyl, alkynyl, aryl, heteroaryl, aralkyl, or heteroaralkyl;
m is an integer in the range 1 to about 10; and
n is an integer in the range 0 to 6.

In one embodiment, X is O.

In one embodiment, Z represents —CO₂R′or —B(OH)₂.

In one embodiment, Z represents —CO₂R′, and R′ represents H.

In one embodiment, Z represents —B(OH)₂.

In one embodiment, R¹ and R² are taken together to form a five-membered ring, giving the amino acid residue D-proline.

In one embodiment, X is O; Z is —B(OH)₂; R¹ is H; R² is the side chain of the amino acid residue tryptophan; R³ is H; m is 1; Y is H or an N-terminal protecting group; and n is 0.

In one embodiment, X is O; Z is —B(OH)₂; R¹ is H; R² is the side chain of the amino acid residue tryptophan; R³ is H; m is 1; Y is H; and n is 0.

In one embodiment, X is O; Z is —B(OH)₂; R¹ is H; R² is the side chain of the amino acid residue tryptophan; R³ is H; m is 1; Y is an N-terminal protecting group; and n is 0.

In one embodiment, X is O; Z is —B(OH)₂; R¹ is H; R² is the side chain of the amino acid residue tryptophan; R³ is H; m is 1; Y is Ac; and n is 0.

In one embodiment, X is S.

In one embodiment, Z represents —CO₂R′ or —B(OH)₂.

In one embodiment, Z represents —CO₂R′, and R′ represents H.

In one embodiment, Z represents —B(OH)₂.

In one embodiment, R¹ and R² are taken together to form a five-membered ring, giving the amino acid residue D-proline.

In one embodiment, X is S; Z is —B(OH)₂; R¹ is H; R² is the side chain of the amino acid residue tryptophan; R³ is H; m is 1; Y is H or an N-terminal protecting group; and n is 0.

In one embodiment, X is S; Z is —B(OH)₂; R¹ is H; R² is the side chain of the amino acid residue tryptophan; R³ is H; m is 1; Y is H; and n is 0.

In one embodiment, X is S; Z is —B(OH)₂; R¹ is H; R² is the side chain of the amino acid residue tryptophan; R³ is H; m is 1; Y is an N-terminal protecting group; and n is 0.

In one embodiment, X is S; Z is —B(OH)₂; R¹ is H; R² is the side chain of the amino acid residue tryptophan; R³ is H; m is 1; Y is Ac; and n is 0.

Other examples of FAP specific inhibitors are disclosed in WO 20061058720A2 WO 20061125227A2; Van der Veken, P, De Meester, I, Dubois, V, Soroka, A, Van Goethem, S, Maes, MB, Brandt, I, Lambeir, A M, Chen, X, Haemers, A, Scharpe, S, Augustyns, K. (2008) Inhibitors of dipeptidyl peptidase 8 and dipeptidyl peptidase 9. Part 1: identification of dipeptide derived leads. Bioorganic and Medicinal Chemistry Letters, 18, 4154-8; and Van der Veken, P, Soroka, A, Brandt, I, Chen, Y S, Maes, MB, Lambeir, A M, Chen, X, Haemers, A, Scharpe, S, Augustyns, K, De Meester, I. (2007) Irreversible inhibition of dipeptidyl peptidase 8 by dipeptide-derived diaryl phosphonates. Journal of Medicinal Chemistry, 50, 5568-70.

### III Methods of Treatment

In certain embodiments there is provided a use of a FAP specific inhibitor in one or more of the following applications:
controlling blood glucose level in an individual, especially for lowering an elevated blood glucose level in an individual resulting from food intake;
increasing insulin secretion in an individual;
decreasing glucagon secretion in an individual;
increasing β cell mass and insulin gene expression in an individual;
inhibiting acid secretion and gastric emptying in the stomach in an individual;
decreasing food intake by increasing satiety in an individual.

An individual suitable for treatment with this method may have diabetes or new onset diabetes or related angiopathy or complications thereof, or a pre-diabetic state such as metabolic syndrome, impaired glucose tolerance, impaired fasting glucose or insulin resistance.

Such individuals may have elevated blood glucose levels, or in other words, a fasting blood glucose of 100 mg/dL or greater or a two-hour 75-g oral glucose tolerance test value of 140 mg/dL or greater. Typically, in accordance with the method, blood glucose is lowered so as to achieve a blood glucose level characterised by a fasting blood glucose of less than 100 mg/dL or a two-hour 75-g oral glucose tolerance test values of less than 140 mg/dL.

In this embodiment, the individual may have elevated levels of haemoglobin A1c.

The individual may or may not have hypertension. Generally speaking, hypertension is present when the systolic blood pressure is greater than about 140 mmHg or when the diastolic blood pressure is greater than about 90 mmHg.

In other embodiments there is provided a method of preventing or treating one or more of the following diseases or conditions:
impaired glucose tolerance;
impaired fasting glucose;
insulin resistance;
new onset diabetes mellitus;
metabolic syndrome;
diabetes mellitus;
hepatic steatosis; the method including providing a FAP specific inhibitor to an individual having one or more the above diseases or conditions or, to an individual in need of treatment of one or more of the above diseases or conditions, or to an individual susceptible for one or more of the above diseases or conditions.

Individuals likely to benefit from the above method include but are not limited to individuals with an increased body mass index (greater than 25 kg/m²), patients with a disease characterised by abnormal PPARy function, those at risk of developing a disease characterised by abnormal PPARy function, individuals at risk of developing hypertension, patients with a disturbance of lipid metabolism (such as for example triglycerides >150 and/dl or low density lipoprotein >130 mg/dl cholesterol or total cholesterol >200 mg/dl or high density lipoprotein cholesterol <60 mg/dl), patients with renal dysfunction (such as for example, those with a plasma creatinine level, greater than 1.5 mg/dl in men and 1.4 mg/dl in women), and patients with first-degree relatives who are suffering or have suffered from diabetes. Other individuals include those having a high fat diet, especially a high saturated fat diet, for example a diet including above average recommended daily intake of saturated fat per day. In certain embodiments the individual receiving said FAP specific inhibitor is one having an increased level of FAP expression or production as compared with a normal individual (i.e. one who does not have a disease or condition mentioned herein). The increased level of FAP expression or production may be 1.5, 2, 5, 10, 20 or 100 fold or more than observed in a normal individual. In certain embodiments the increased expression or production of FAP is as a consequence of diet, especially a high fat diet.

In certain embodiments there is provided a method of preventing the development of new onset diabetes mellitus in an individual including:
selecting an individual having a pre-diabetic state; and
administering a FAP specific inhibitor to the selected individual.

Typically the pre-diabetic state consists of one or more of metabolic syndrome, impaired glucose tolerance, impaired fasting glucose and insulin resistance.

FAP specific inhibitors described herein are used to treat or prevent one or more of the above described diseases or conditions and may be administered as soon as possible after diagnosis. Early administration is especially preferred to prevent an individual from advancing from a pre diabetic state to new onset diabetes or to prevent advancement from new onset diabetes to diabetes. Even in cases of active diabetes, early administration may be particularly useful for minimising or controlling angiopathy and related complications.

The FAP specific inhibitors are used in accordance with the invention in a therapeutically effective amount. The amount of inhibitor administered depends on the subject being treated, on the subject's weight, the severity of the affliction, the manner of administration and the judgment of the prescribing physician. As a general proposition, the initial pharmaceutically effective amount of the inhibitor administered per dose will be in the range of about 0.01-100 mg/kg, namely about 0.1 to 20 mg/kg of patient body weight per day, with the typical initial range of compound used being 0.3 to 15 mg/kg/day. The therapy can be applied while symptoms are detectable or even when they are not detectable. Generally the therapy is applied when the particular symptoms of the given disease or condition become of concern. In certain embodiments the therapy is applied at least once daily for a period of time over which fasting glucose or glucose tolerance measurements would otherwise remain outside normal ranges in the absence of therapy. The therapy can be provided alone or in combination with other drugs.

A therapeutically effective amount of the inhibitor can provide therapeutic benefit without causing substantial toxicity. Toxicity of the inhibitor can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Inhibitors exhibiting high therapeutic indices are preferred (see e.g., Fingl et al., 1975, in: The Pharmacological Basis of Therapeutics, Ch. 1, p. 1).

The response of an individual to treatment can be monitored by determining fasting glucose or glucose tolerance according to standard techniques. Typically, in accordance with the method, blood glucose is lowered so as to achieve a blood glucose level characterised by a fasting blood glucose of less than 100 mg/dL or a two-hour 75-g oral glucose tolerance test values of less than 140 mg/dL. In other embodiments response to treatment may include determining the other factors relevant to pre diabetes, new onset diabetes or active diabetes including blood pressure, body mass index, PPARy function, lipid metabolism and renal function.

The FAP specific inhibitor may be administered by any route appropriate to the disease or condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary, and intranasal. It will be appreciated that the preferred route may vary with for example the condition of the recipient.

### IV Compositions and formulations

In other embodiments there is provided a use of a FAP specific inhibitor in the manufacture of a medicament in one of the above described applications.

Typically in the above described embodiments the FAP specific inhibitor is provided as the only active pharmaceutical principle selected for the treatment or prevention of the disease or condition.

In certain embodiments the FAP specific inhibitor may be provided in the form of a composition in which other compounds are provided as diluents, carriers, excipients or like compounds. In these forms of the invention the composition consists essentially of the FAP specific inhibitor as an active ingredient.

Acceptable diluents, carriers, excipients, and stabilizers are nontoxic to recipients at the dosages and concentrations employed, and include buffers such as phosphate, citrate, and other organic acids; antioxidants including ascorbic acid and methionine;

preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as plasma albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counterions such as sodium; metal complexes (e.g. Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable, examples of sustained-release preparations include semipermeable matrices of solid hydrophobic polymers containing the N-acylated dipeptide proline boronate compound, which matrices are in the form of shaped articles, e.g. films, or microcapsules. Examples of sustained-release matrices include polyesters, hydrogels (for example, poly(2-hydroxyethyl-methacrylate), or polyCvinylalcohol)), polylactides, copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid.

Compounds of the invention may be prepared for various routes and types of administration. A FAP specific inhibitor having the desired degree of purity is optionally mixed with pharmaceutically acceptable diluents, carriers, excipients or stabilizers (Remington's Pharmaceutical Sciences (1980) 16th edition, Osol, A. Ed.), in the form of a lyophilized formulation, milled powder, or an aqueous solution. In general the formulations are prepared by uniformly and intimately bringing into association the active ingredient with liquid carriers or finely divided solid carriers or both, and if necessary, shaping the product. Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment. The inhibitory compound for use herein is preferably sterile. The compound ordinarily will be stored as a solid composition, although lyophilized formulations or aqueous solutions are acceptable.

The pharmaceutical composition (or formulation) for application may be packaged in a variety of ways depending upon the method used for administering the drug. Generally, a kit or article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

Formulations of FAP specific inhibitors suitable for oral administration may be prepared as discrete units such as pills, capsules, cachets or tablets each containing a predetermined amount of the FAP specific inhibitor.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

Tablets, troches, lozenges, aqueous or oil suspensions, dispersible powders or granules, emulsions, hard or soft capsules, e.g. gelatin capsules, syrups or elixirs may be prepared for oral use. Formulations of a FAP specific inhibitor intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. Excipients may include, but are not limited to, calcium carbonate, sodium carbonate, lactose, calcium phosphate, sodium phosphate, mannitol, crospovidone, polysorbate 80, hydroxypropyl methylcellulose, colloidal silicon dioxide, microcrystalline cellulose, sodium starch glycolate, simethicone, polyethylene glycol 6000, sucrose, magnesium carbonate, titanium dioxide, methylparaben, and polyvinyl alcohol. Excipients may also include granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Topical ointments or creams may contain the active ingredient(s) in an amount of, for example, 0.075 to 20% w/w. When formulated in an ointment, the active ingredients may be employed with either a paraffinic or a water-miscible ointment base. Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

An oily phase of an emulsion may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

Aqueous suspensions contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The pharmaceutical composition of a FAP specific inhibitor may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable) volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents.

Formulations suitable for vaginal administration may be presented as pessaries, tampons, creams, gels, pastes, foams or spray formulations containing in addition to the active ingredient such carriers as are known in the art to be appropriate.

The formulations may be packaged in unit-dose or multi-dose containers, for example pills, sealed ampoules, vials, and blister packs. Formulations may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient. The invention further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

In other embodiments the FAP specific inhibitor may be provided in the form of a composition including other active principles for treatment or prevention of the disease or condition. Examples of these include glitazones (e.g. trogliazone, pioglitazone, englitazone, rosiglitazone, and the like); biguanides such as: phenformin and metformin; protein tyrosine phosphatase 1-B inhibitors; insulin or insulin mimetics; and sulfonylureas such as tolbutamide and glipizide; and a glucosidase inhibitors, examples of which include miglitol, voglibose and acarbose.

In certain embodiments where a FAP specific inhibitor and another active principle is to be provided for treatment or prevention of a disease or condition, the inhibitor and other active principle may be provided simultaneously, in which case they may be formulated in a composition as described above. Alternatively they may be provided from separate aliquots and administered simultaneously.

In other embodiments a FAP specific inhibitor and another active principle to be provided for treatment or prevention of a disease or condition may be provided sequentially, for example with the FAP specific inhibitor provided before the other active principle or vice versa. In these embodiments the inhibitor and the other principle are provided from separate aliquots.

The FAP specific inhibitor may be combined in a pharmaceutical combination formulation, or dosing regimen as combination therapy, with a second compound that can be used to treat a disorder or a complication stemming from abnormal blood glucose level of control. The second compound of the pharmaceutical combination formulation or dosing regimen preferably has complementary activities to the FAP specific inhibitor of the combination such that they do not adversely affect each other. Such molecules are suitably present in combination in amounts that are effective for the purpose intended.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities.

Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, i.e. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, i.e. serially, whereas in combination therapy, effective dosages of two or more active ingredients are administered together.

In other embodiments there is provided a kit for use in one of the above described embodiments, the kit including:
a container holding a FAP specific inhibitor;
a label or package insert with instructions for use.

In certain embodiments the kit may contain one or more active principles for treatment or prevention of the disease or condition described above.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a FAP specific inhibitor or formulation thereof which is effective for treating the condition and may have a sterile access port (for example the container may be an intravenous solution bag or a vial having a stopper pierceable by a hypodermic injection needle). At least one active agent in the composition is a FAP specific inhibitor. The label or package insert indicates that the composition is used for treating the condition of choice, such as diabetes or new onset diabetes. In one embodiment, the label or package insert includes instructions for use and indicates that the composition comprising the FAP specific inhibitor can be used to treat a disorder or a complication stemming from abnormal blood glucose level of control.

The kit may comprise (a) a first container with a FAP specific inhibitor; and (b) a second container with a second active principle contained therein. The kit in this embodiment of the invention may further comprise a package insert indicating that the inhibitor and other active principle can be used to treat a disorder or a complication stemming from abnormal blood glucose level of control. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

### Example 1

### Aim

To determine whether gene knock out (gko) mice strains of DPIV and FAP are protected from a high-fat-diet induced (HFD) obesity and liver steatosis.

### Methods

C57BL/6 (WT; wildtype) (n=10), DPIV^{-/-} (n=11,12) and FAP^{-/-} (n=11) mice of 6-8 week old were obtained from the Animal Resources Centre (ARC, Perth, West Australia). The animals were cared for in accordance with protocols approved by Animal Ethics Committees of the University of Sydney. The mice were fed either the High Fat Diet (HFD), purchased from Specialty Feeds (23% fat plus 0.19% cholesterol, Cat. No. SF03-020, Perth) with water supplemented with 5% fructose (Sigma) or ad libitum chow (control diet). The mice were monitored for weight gain over the 12 weeks of diet. At 12 weeks, liver, spleen, fat and plasma were collected for further analyses. Standard liver function tests including plasma levels of Alanine Aminotransferase (ALT), Aspartate Aminotransferase (AST), Alkaline phosphatase (ALP), albumin, total protein and total bilirubin were performed by Royal Prince Alfred Hospital Clinical Biochemistry on these mouse plasma.

The Specialty Feeds HFD was chosen following a comparison with HFD from Research Diets (New Jersey) that showed little difference in WT weight gain (Appendix 1).

### Statistical Analysis

The data are presented as mean±SEM. Statistical analysis was performed using the Prism 5 software, applying One-way ANOVA followed by the Newman-Keuls multiple comparison test, except when using Student's t test. P values <0.05 were considered significant. Correlation graph was generated with Microsoft Office Excel software.

### Results

DPIV^{-/-} and FAP^{-/-} mice on HFD gained significantly less weight and adipose tissue over 12 weeks of HFD compared to WT mice on HFD. The same outcome was observed in a separate experiment in which feeding was extended a further 8 weeks to 20 weeks.

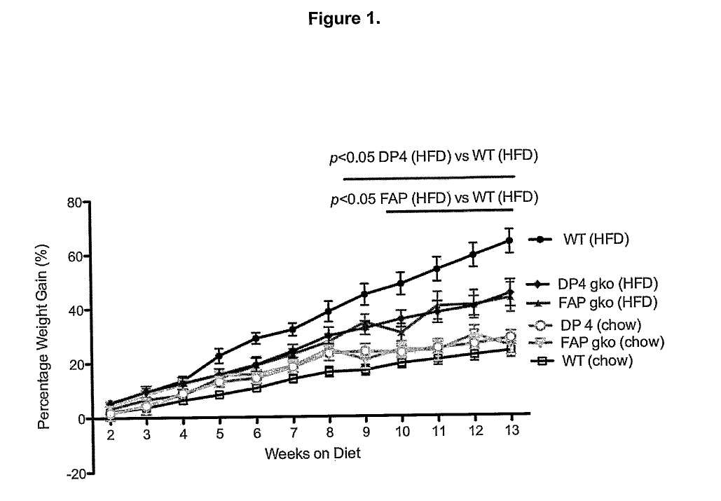

Female WT, DPIV^{-/-} and FAP^{-/-} mice were fed an ad libitum chow diet (chow) or high fat diet (HFD-23% fat diet supplemented with 5% fructose in water ad libitum) for 12 weeks. Groups had n=10 to 12. As shown in FIG. 1, DP4^{-/-} and FAP^{-/-} mice on HFD gained significantly less body weight when compared to the WT mice on HFD. DP4^{-/-} and FAP^{-/-} mice exhibited similar weight gains.

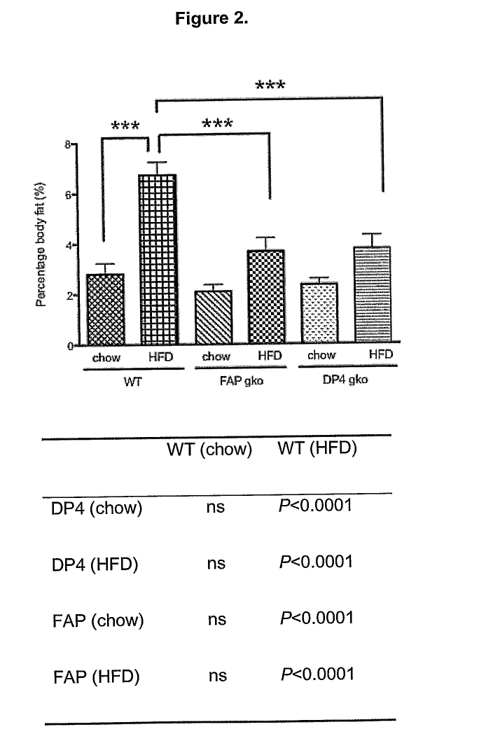

FIG. 2 shows adiposity index, calculated by total body fat/body weight ratio of wildtype (WT), FAP^{-/-} and DP4^{-/-} mice after 12 weeks of diet. WT mice had a significantly greater percentage of total fat content after HFD compared to chow. Fed on HFD, FAP and DP4 gko mice accumulated significantly less fat compared to WT mice.

### Both DP4 and FAP gko Mice Exhibited Less HFD-Induced Liver Damage

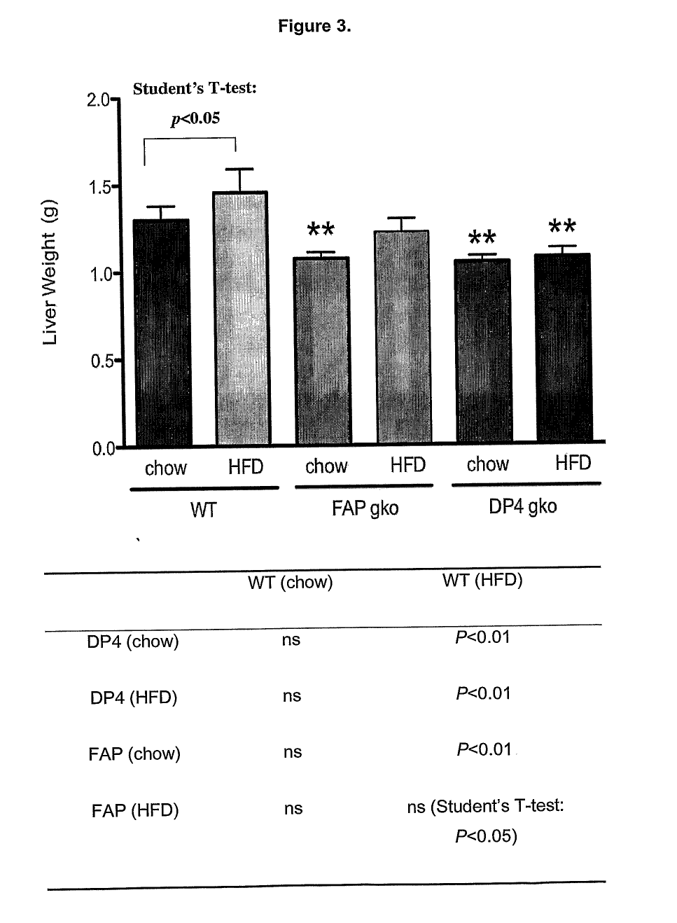

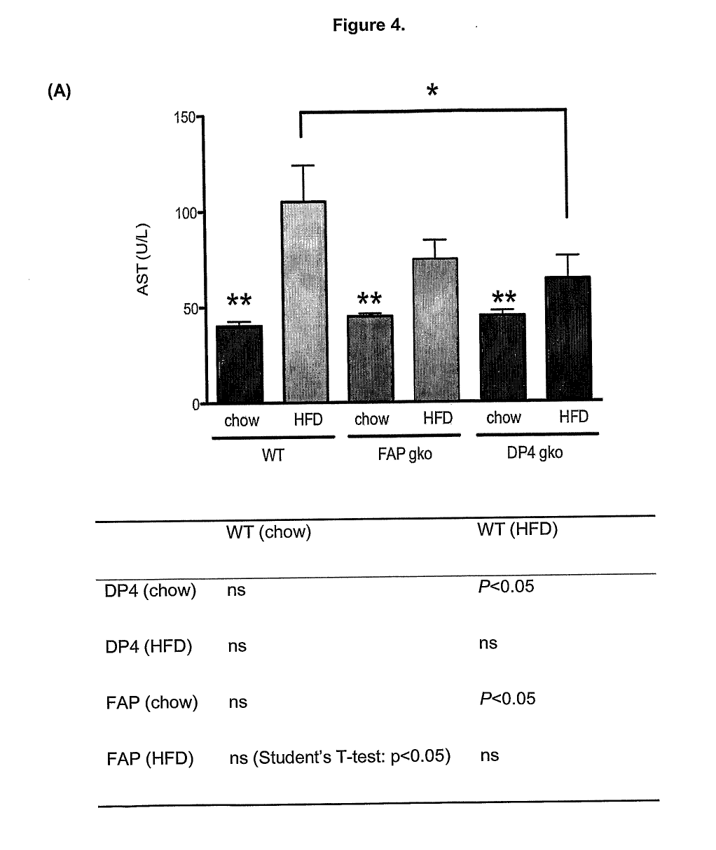

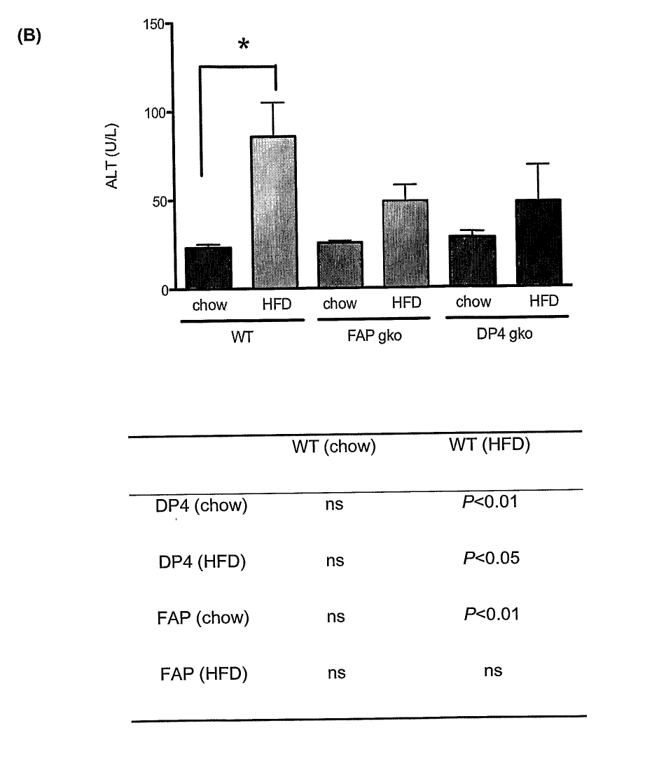

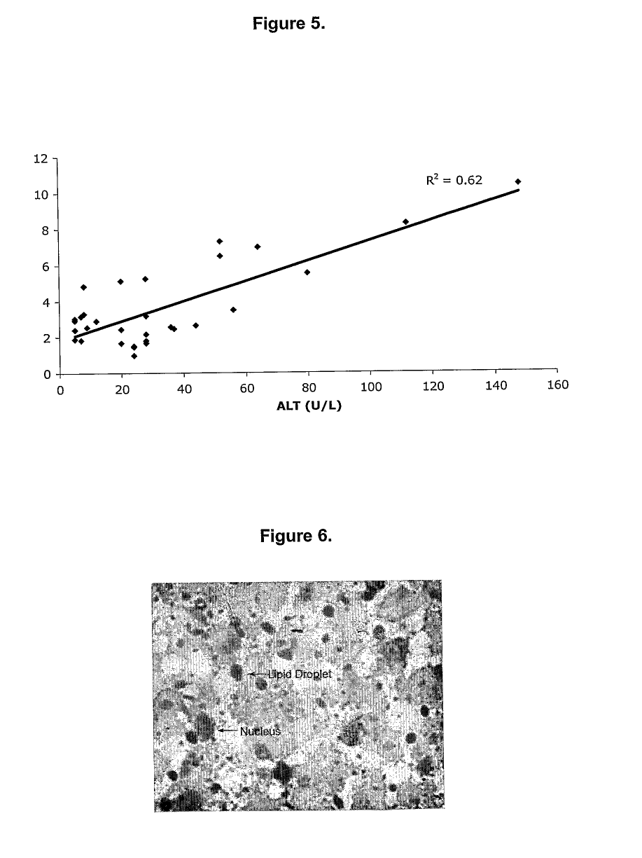

HFD has been associated with hepatic steatosis, which impairs liver function. To determine whether lipid accumulation was evident in mice fed a HFD, the livers were weighed after 12 weeks of diet (FIG. 3). WT mice on HFD exhibited significantly greater liver weight compared to WT mice on chow while HFD did not cause significant increase in liver weight in DP4 and FAP gko mice. This observation suggests that DP4 and FAP inhibition reduced HFD-induced lipid accumulation in liver. To test for presence of hepatic damage, liver functions were examined by measuring the plasma levels of Alanine Aminotransferase (ALT), Aspartate Aminotransferase (AST), Alkaline phosphatase (ALP), albumin, total protein and total bilirubin. ALT and AST are standard tests performed to measure tissue damage. Elevated levels of ALT indicate specific tissue damage to the liver whereas AST levels measure the extent of tissue damage on liver as well as other tissues including heart and kidney. Both ALT and AST levels were significantly elevated in WT mice fed on HFD compared to WT mice on chow, suggesting HFD induced liver/tissue damage. Interestingly, HFD did not increase ALT or AST levels in FAP gko and DP4 gko mice (FIG. 4). AST level of DP4 gko mice on HFD was significantly lower compared to that of WT on HFD, suggesting that less HFD-induced-hepatic damage occurred in DP4 gko mice. HFD did not induce physiologically aberrant levels of nonfasting plasma glucose, ALP, total bilirubin, albumin across the three groups (Appendices). FIG. 5. shows the correlation of ALT with adiposity index (R²=0.62), indicating that the increase in body fat may elevate ALT. FIG. 6 shows extensive lipid droplet deposition in hepatocytes in the liver of an obese mouse.

### Discussion & Conclusion

The association of obesity and liver steatosis with type 2 diabetes has been recognized for decades. The major basis for this association is that insulin resistance is fundamentally the common problem in obesity, liver steatosis and type 2 diabetes. Given the acknowledged beneficial roles of incretins to increase biosynthesis and secretion of insulin and in improving β-cell function, there are therapeutic interests in developing degradation resistant incretin analogues or enzyme inhibitors so to lengthen the half life of bioactive incretin for the treatment of Impaired Glucose Tolerance/Insulin Resistance/Type II diabetes. Obesity has been associated with many other ailments including heart disease and cancer.

DPIV gko mice are protected from high fat diet-induced obesity and fatty liver (2). In addition, injecting DPIV resistant GLP-1 analogue, exendin 4 (4), into ob/ob mice diminishes liver steatosis (3), suggesting the role of DPIV in lipid metabolism. The current study demonstrated consistent data on the protective role of DPIV inhibition from HFD-induced obesity. The key findings are that:
FAP^{-/-} mouse strain was as protected from obesity as the DPIV^{-/-} strain under a high-fat, high sugar metabolic load
DPIV^{-/-} and FAP^{-/-} mice were protected from HFD-associated hepatocyte damage

The role of FAP in protection from obesity probably occurs by enzymatic mechanisms. For example, enzyme inhibition might mimic gene deficiency with the mechanism likely to be via enhancing incretin activities.

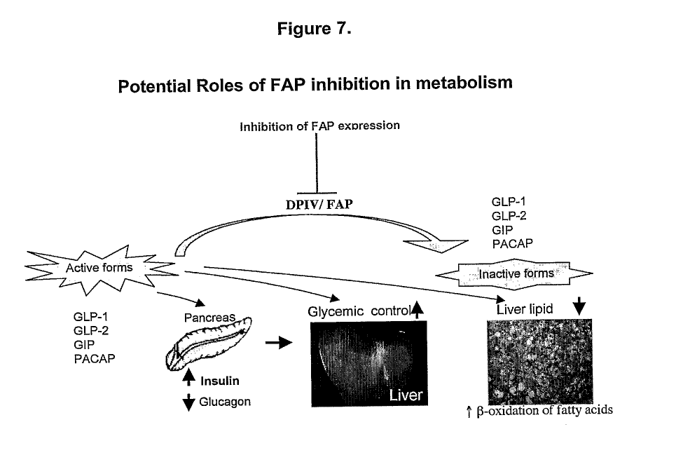

In addition, the effects of FAP inhibition on other various substrates may influence carbohydrate and lipid metabolism (FIG. 7). For example, FAP may well regulate the activity of PACAP38, a regulator of lipid and carbohydrate metabolism.

### Example 2

### Aim

To determine plasma glucose levels of FAP gko mice.

### Methods

### Mice

Female mice of C57BL/6 (WT), and DPIV and FAP gko on C57BL/6 genetic background were housed in Centenary Institute under University of Sydney animal ethics committee approvals.

### Intraperitoneal Glucose Tolerance Test (IPGTT)

Mice aged 8-12 weeks (n=6) were fasted over night before receiving intraperitoneal administration of 4 g of D-glucose per kg body weight in saline (0.9% NaCl). Blood samples of conscious mice were collected from the tail vein at 0, 30, 60, 120 and 180 min. Blood glucose concentration was determined on Accucheck Performa (Roche Diagnostic) (9, 14).

Oral Glucose Tolerance Test (oGTT)

Mice aged 6-8 weeks (n=6) were fed ad libitum either chow or high fat diet (HFD) (23% fat plus 0.19% cholesterol, Cat No. SF03-020, Specialty Feeds, Perth, West Australia) with water supplemented with 5% fructose (Sigma, USA) for 14 weeks. The mice were then fasted for 5 hours before oral administration of 2 g of D-glucose per kg body weight in saline (0.9% NaCl). Blood samples were collected and assayed as described above.

### Realtime RT-PCR

Total RNA was extracted from frozen mouse livers (n=5 per group) using Trizol (Invitrogen) according to the manufacturer's specification.

The mRNA expression level of SCD1 and ACC were then measured using Taqman Gene expression assays (Mm00772290_m1 and Mm01304276_m1, Assays-on-Demand, Applied Biosystems) according to the manufacturer's specification. The reaction mixture (10 μl) contained 5 μl of universal master mix, 0.5 μl of Taqman probe, 3.5 μl of H₂O and 1 μl of cDNA. The cycling parameter was 95° C. for 10 minutes followed by 35 cycles of 95° C. for 15 sec, 60° C. for 1 min. 18s RNA was quantified in all cDNA for normalization. The standard aspects of this method have been published (16).

### Statistical Analysis

The data are presented as mean ±SEM. Student's T test was used to compare groups. P values less than 0.05 were considered significant.

### Results

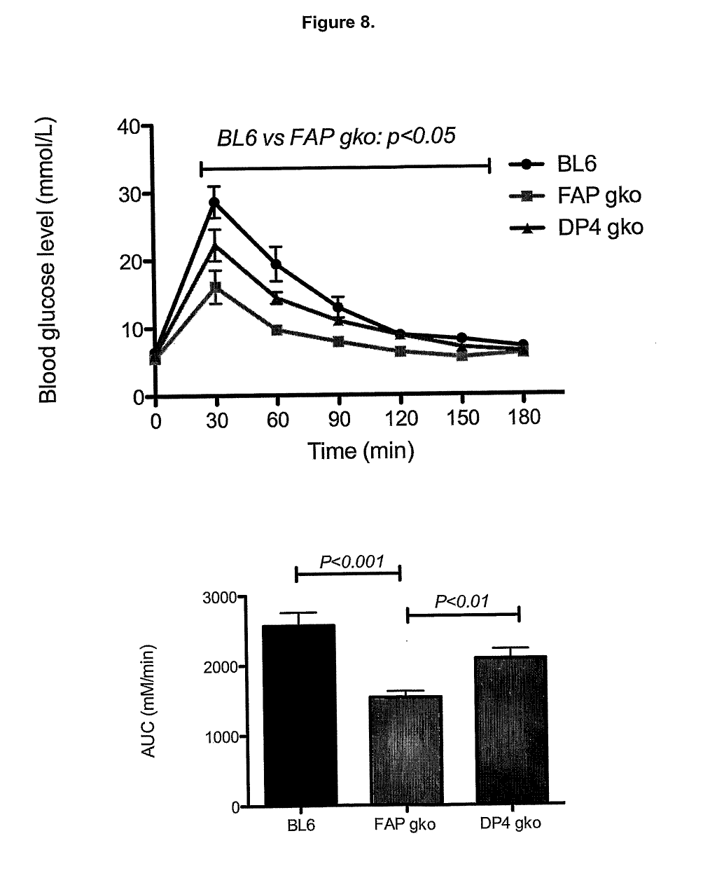

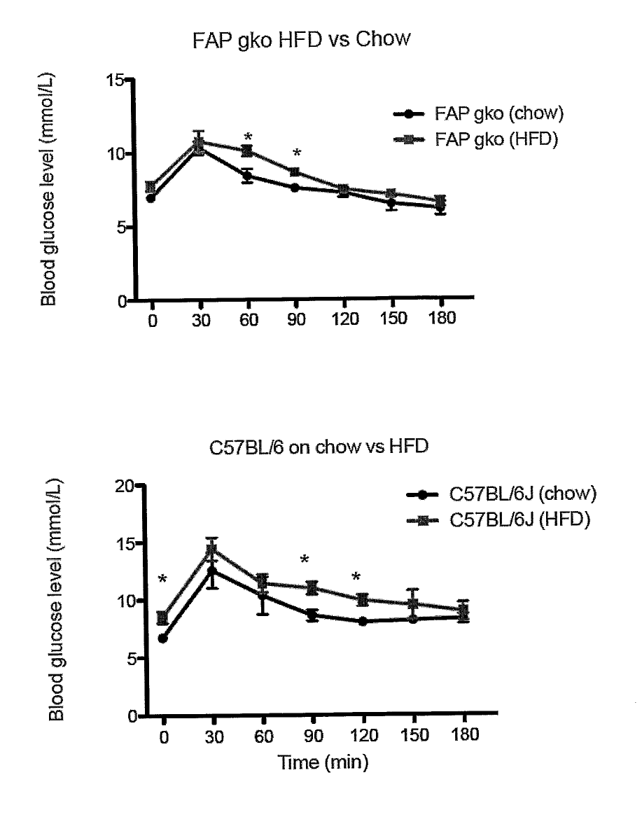

The peak glucose level achieved over the 30 minutes immediately after intraperitoneal administration of glucose (IPGTT) and the AUC were significantly less in FAP gko mice compared to C57BL/6 WT mice (FIG. 8). Plasma glucose levels of FAP gko mice returned close to basal levels within 60 min, which was more rapid than C57BL/6 mice.

Moreover, the fasting glucose level was greater in HFD- than chow-fed only in WT mice, not in FAP gko mice.

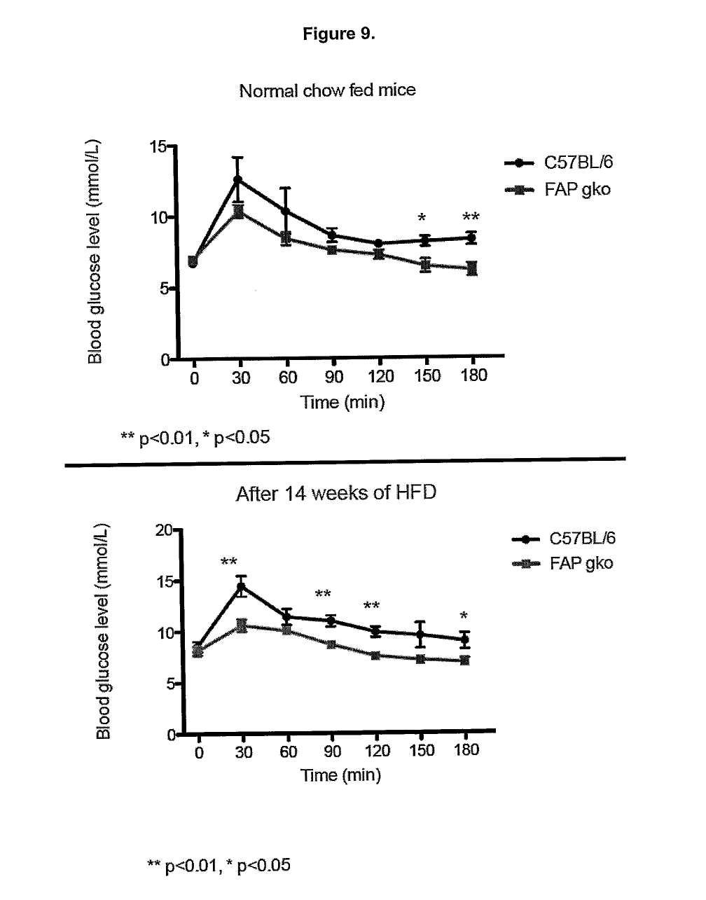

Oral Glucose tolerance test (OGTT) of C57BL/6 and FAP gko mice showed similar observations as IPGTT; improved plasma glucose clearance was evident in FAP gko mice compared to C57BL/6 mice fed either chow or high fat diet for 14 weeks (FIGS. 9(a) and 9(b)).

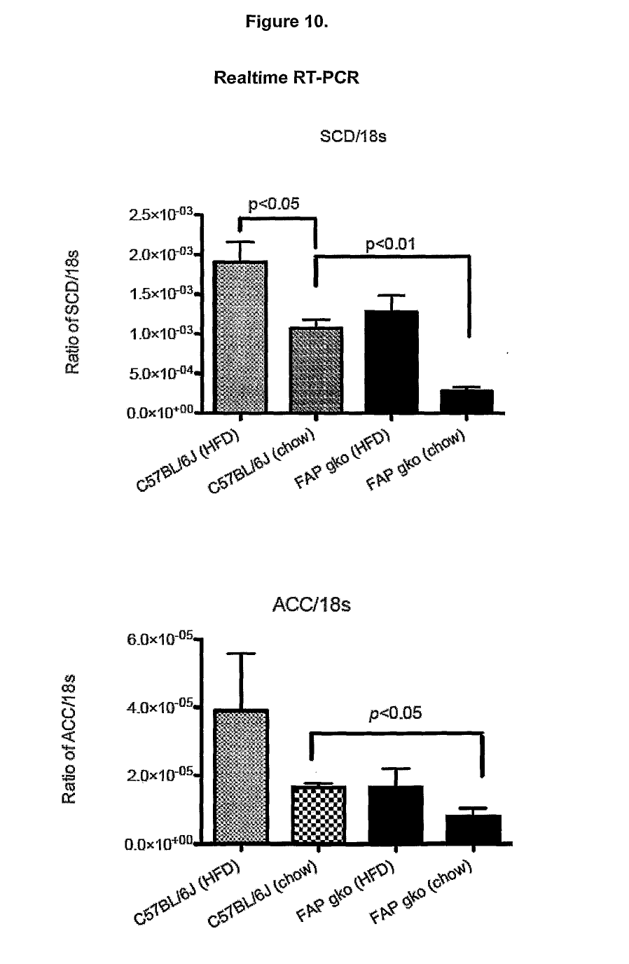

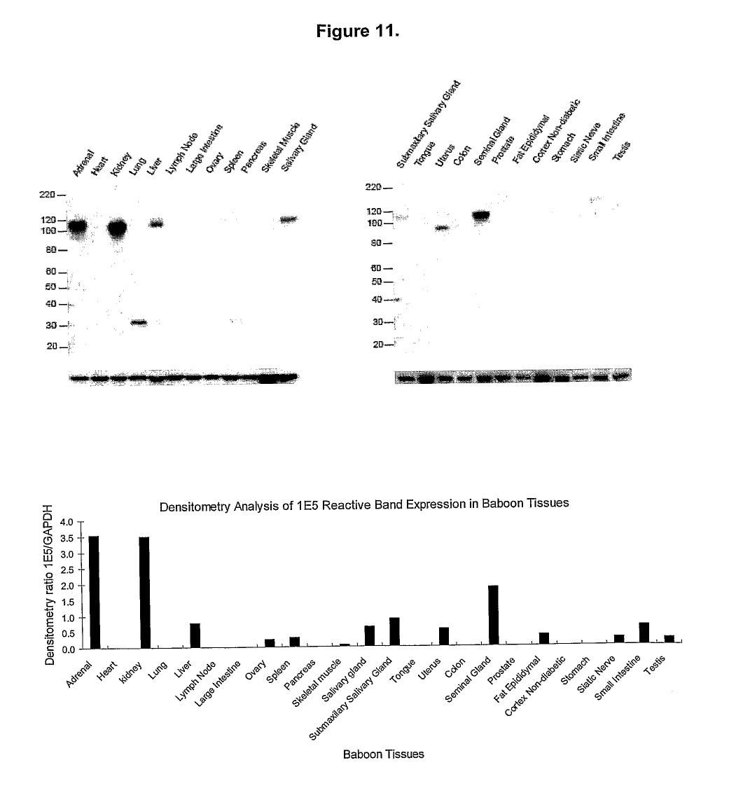

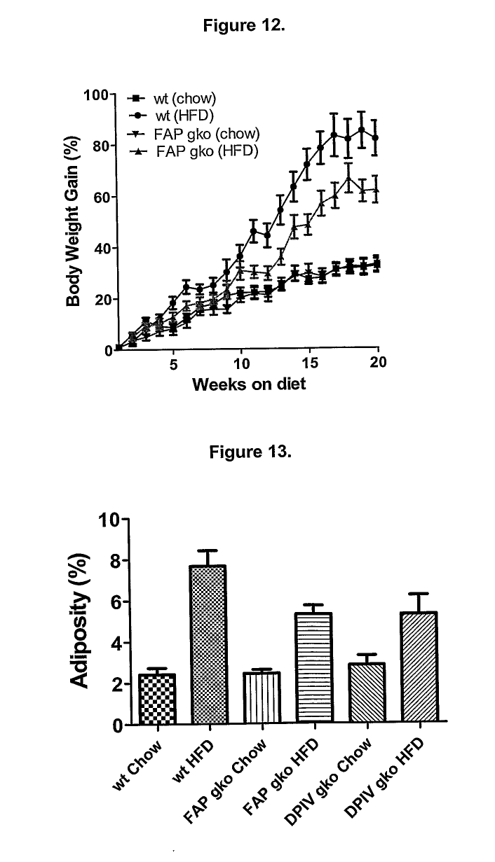

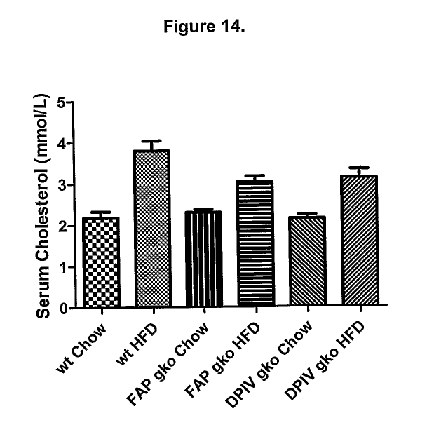

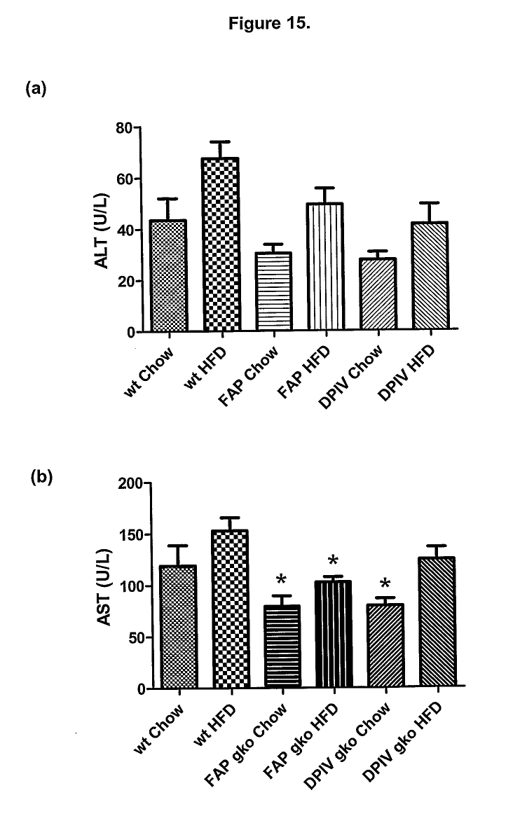

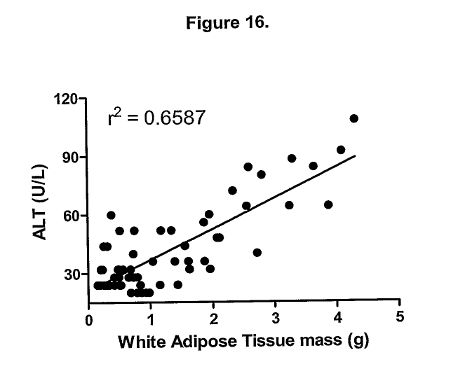

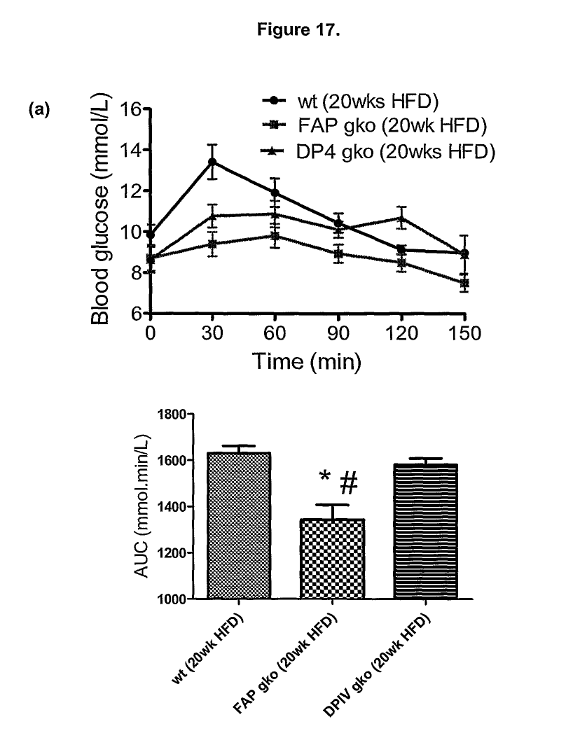

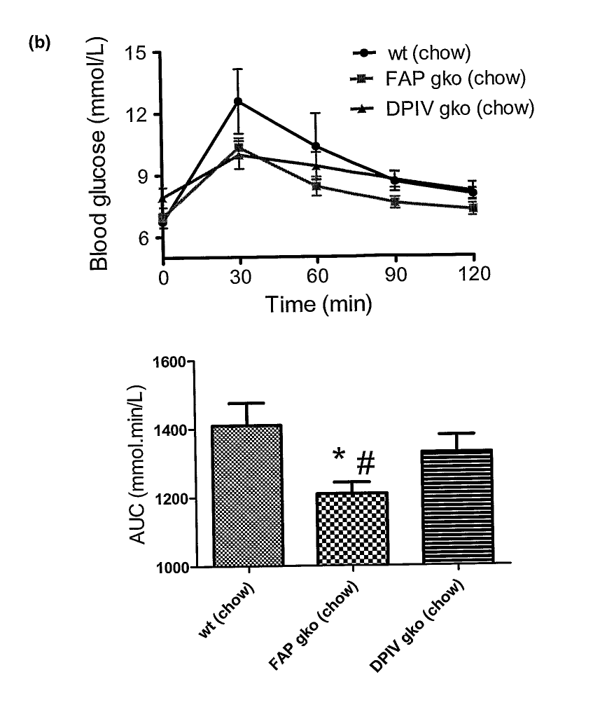

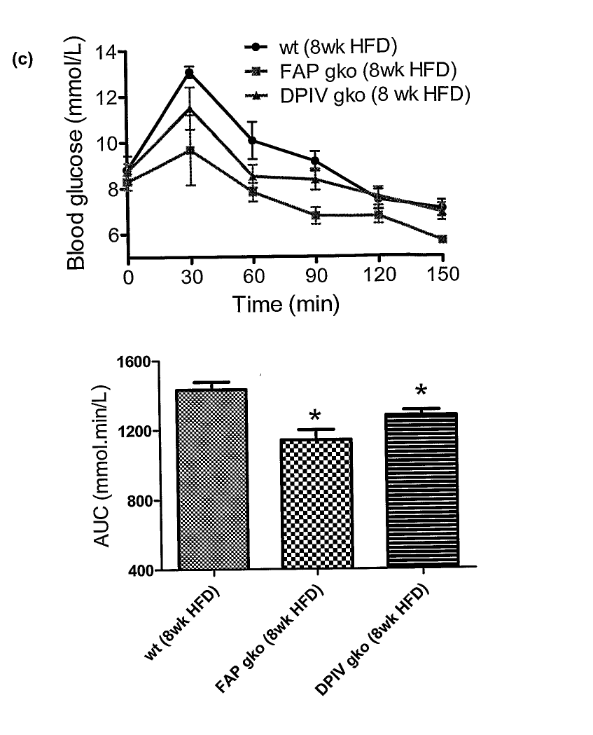

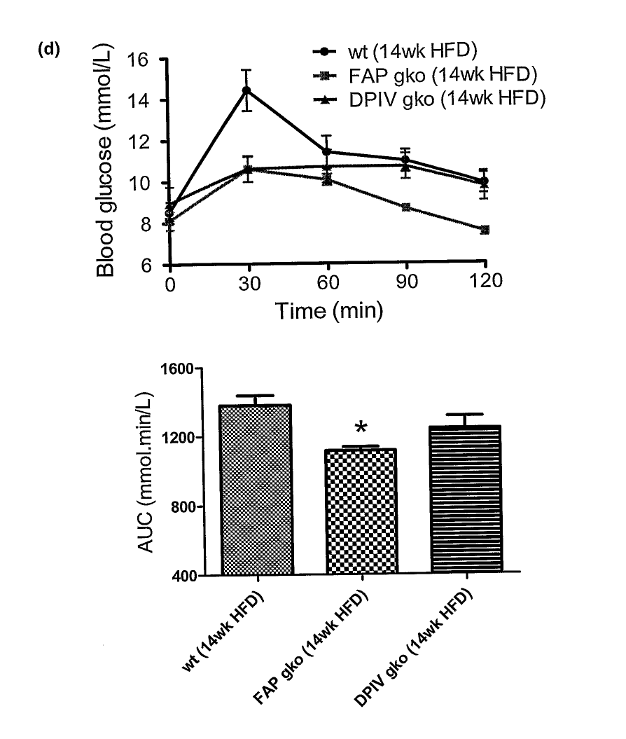

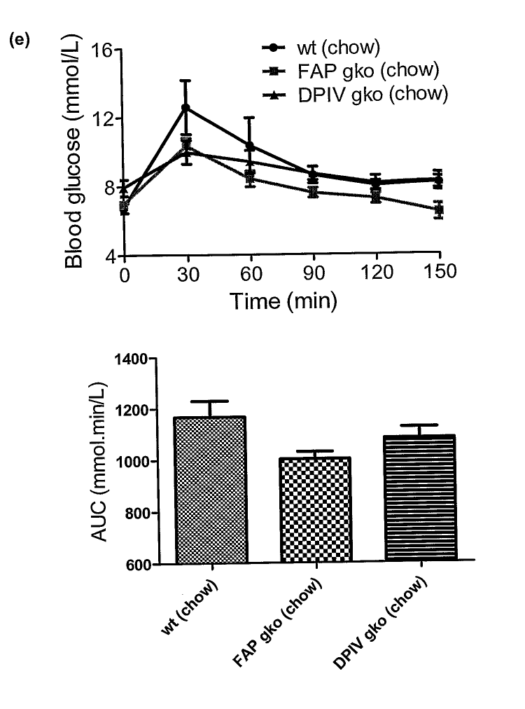

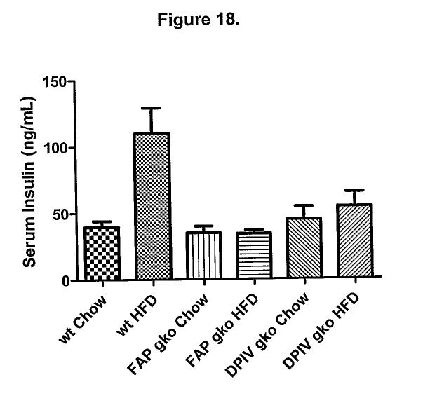

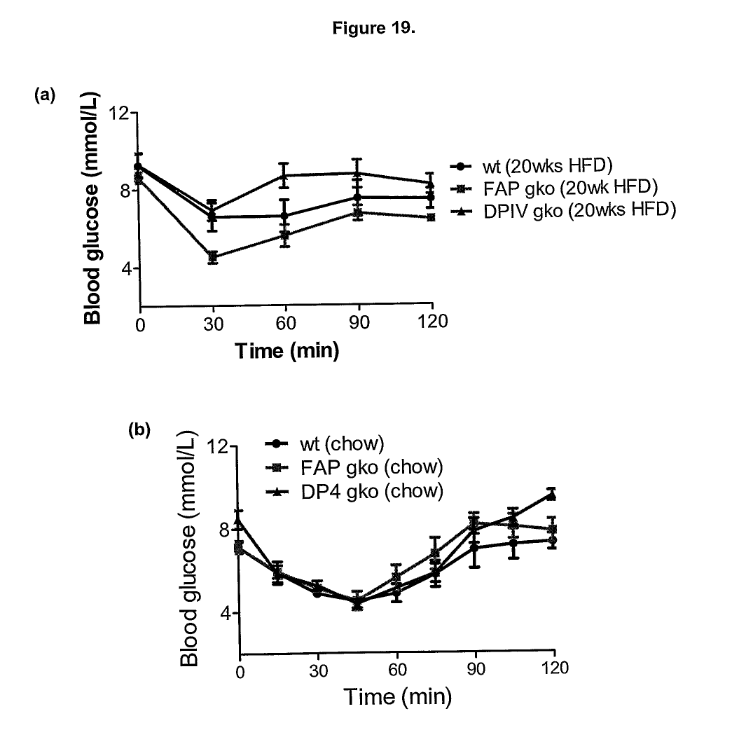

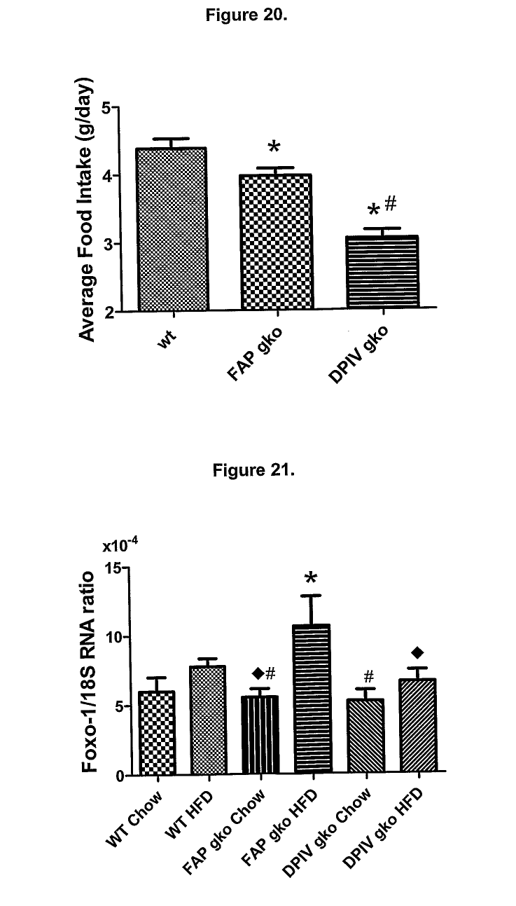

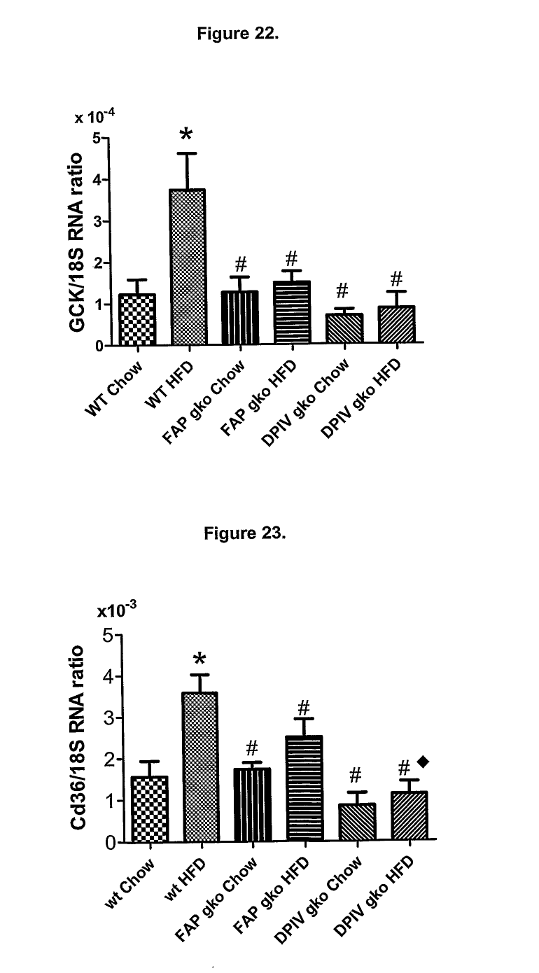

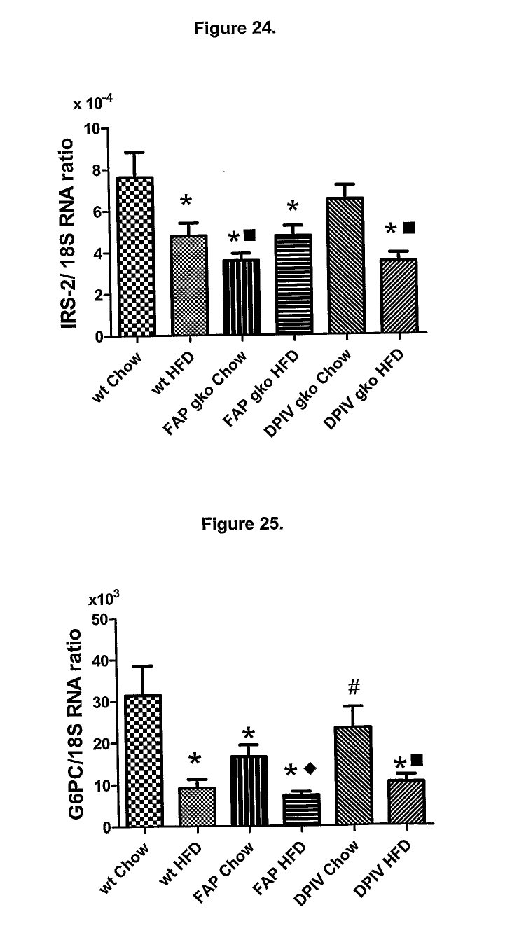

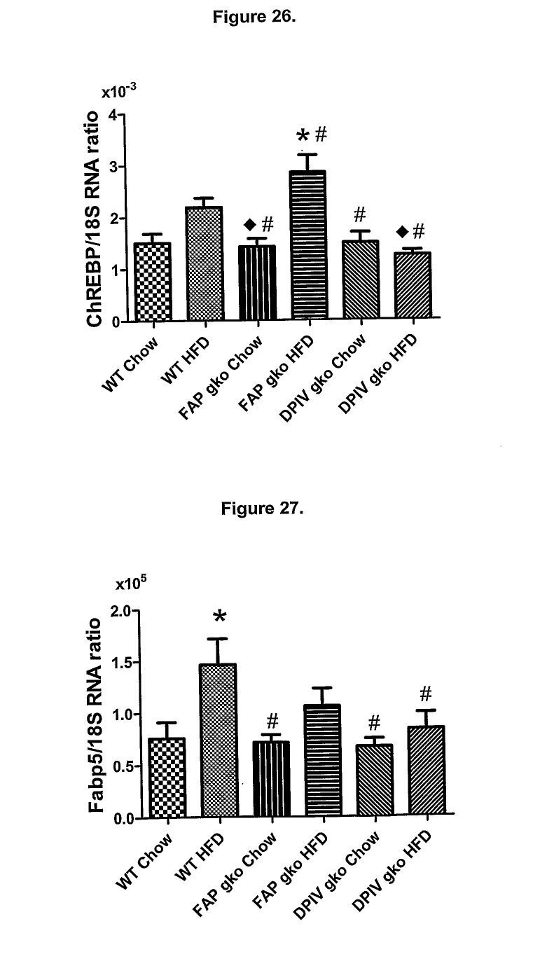

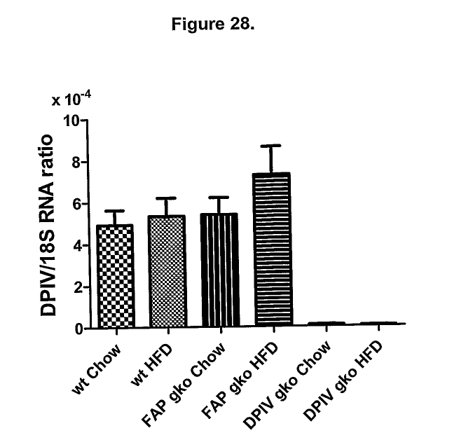

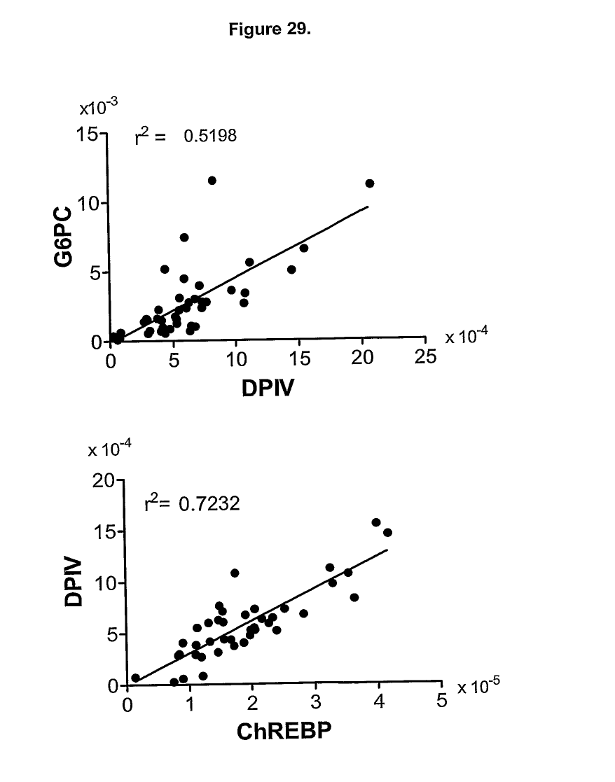

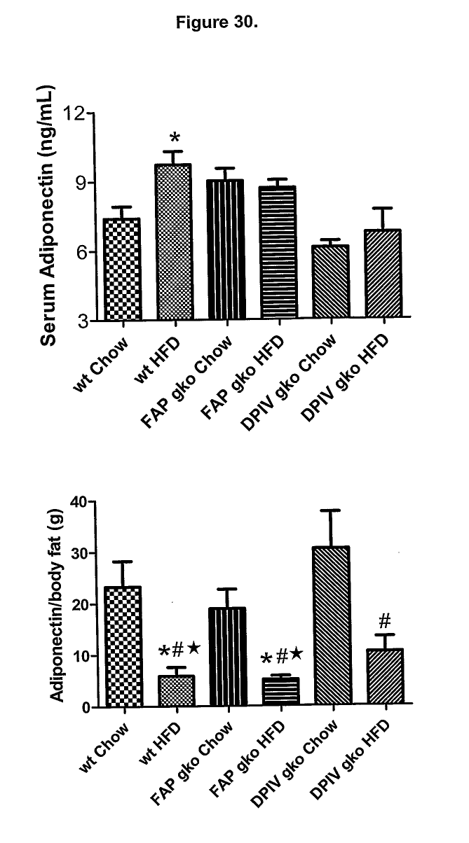

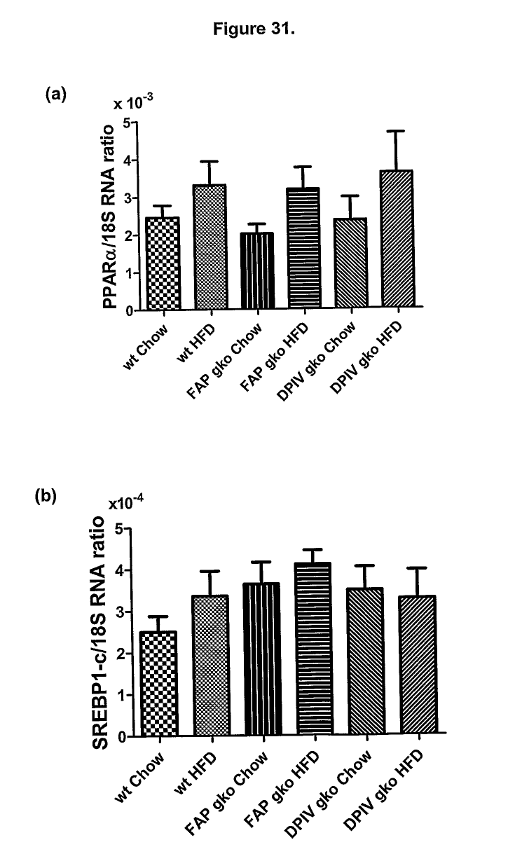

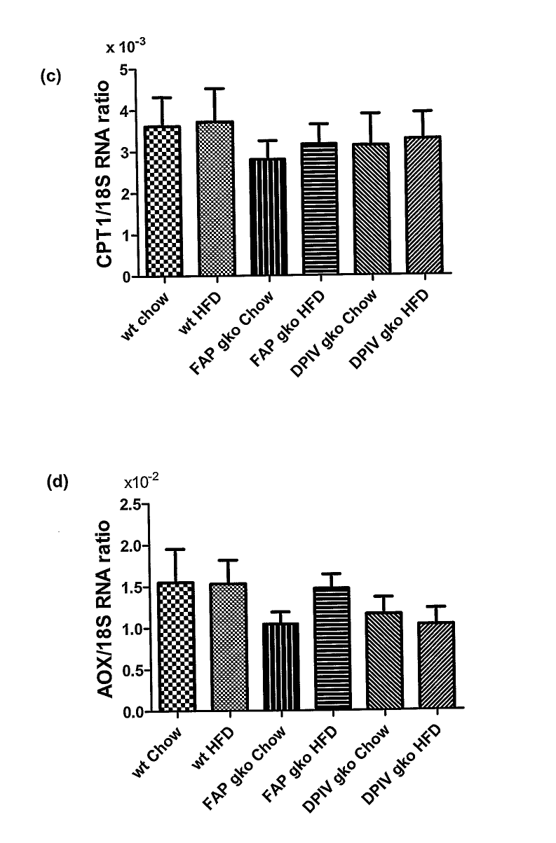

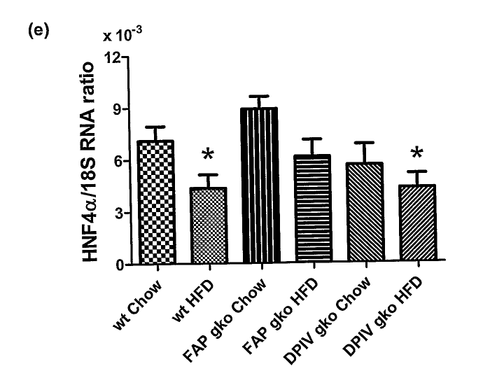

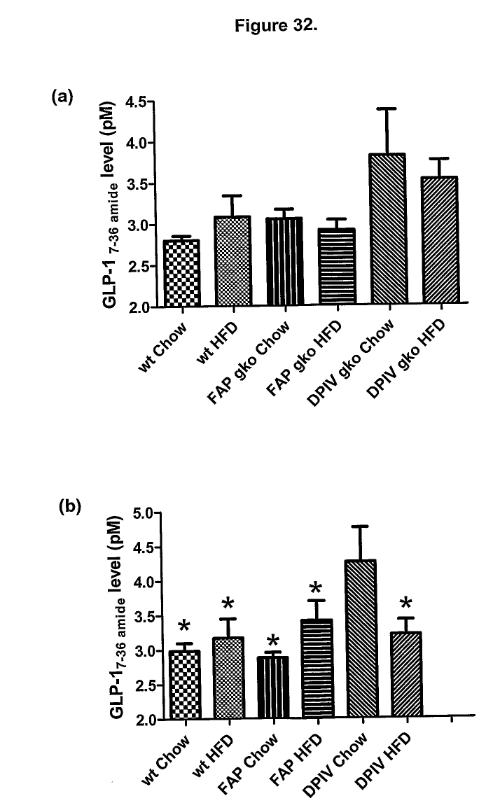

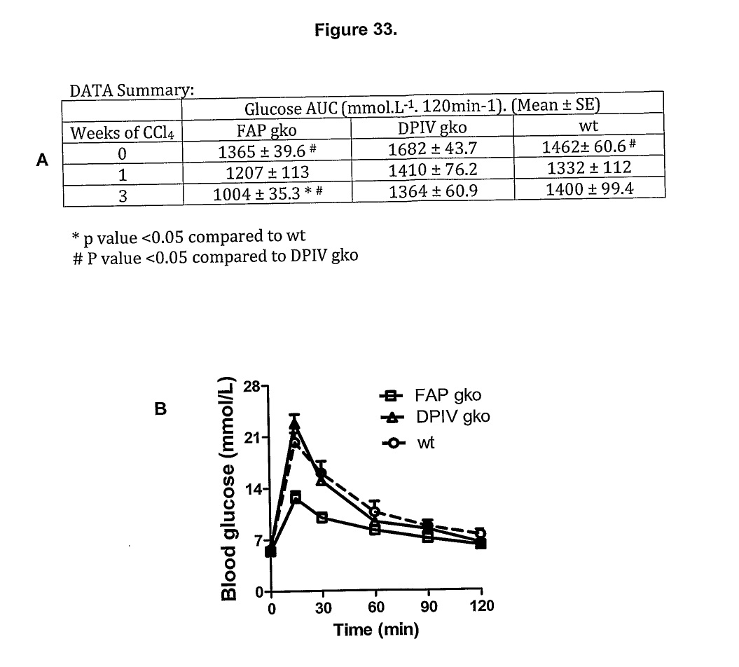

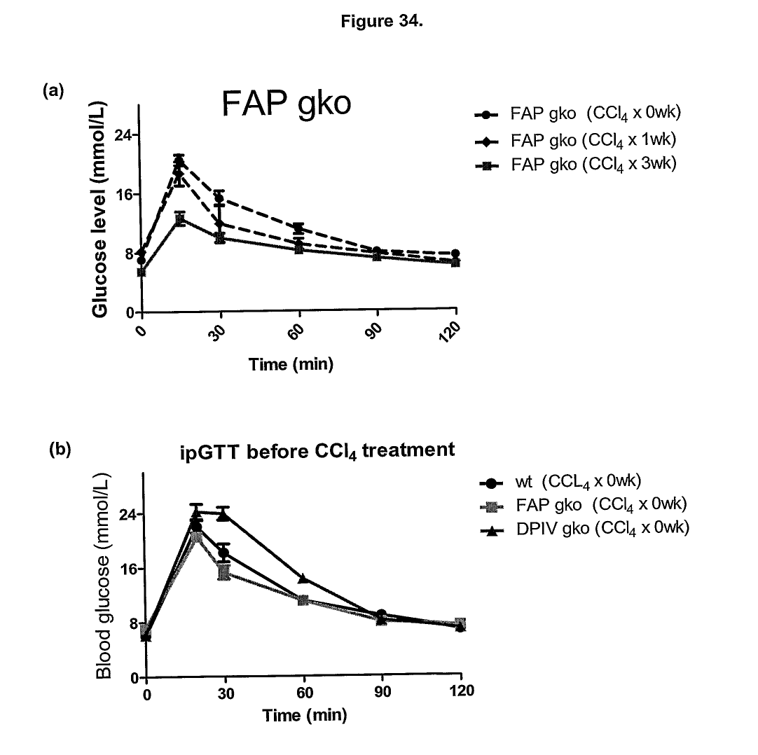

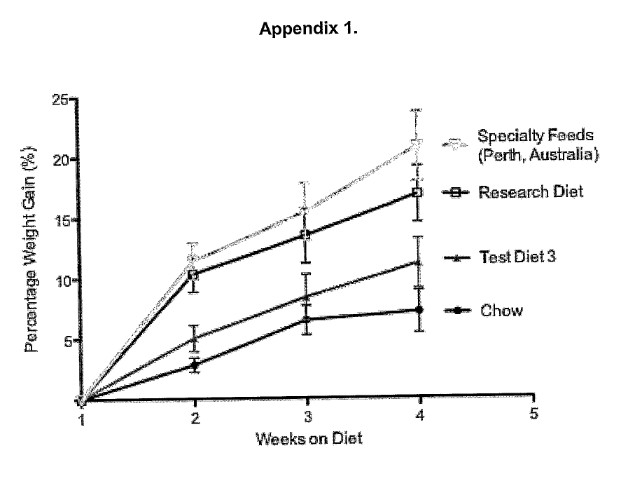

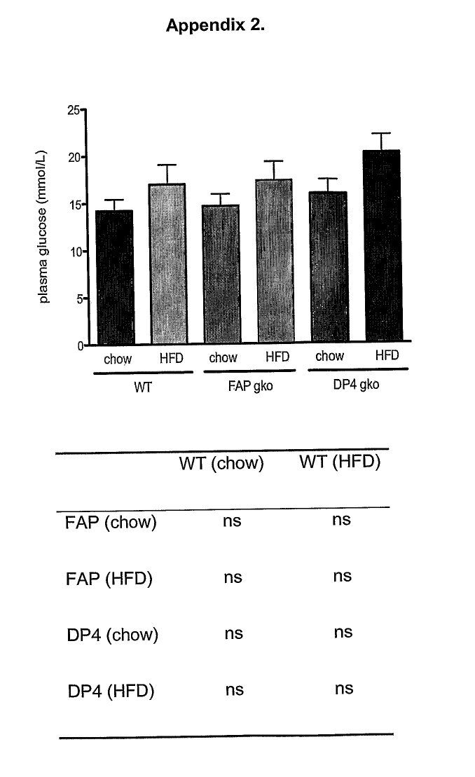

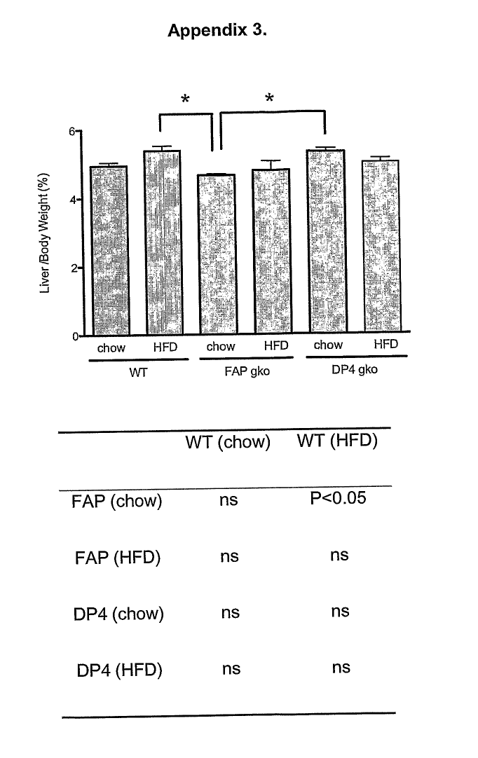

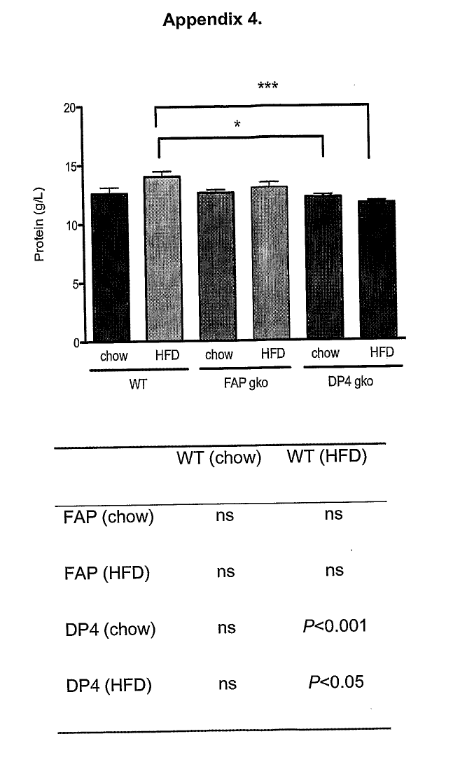

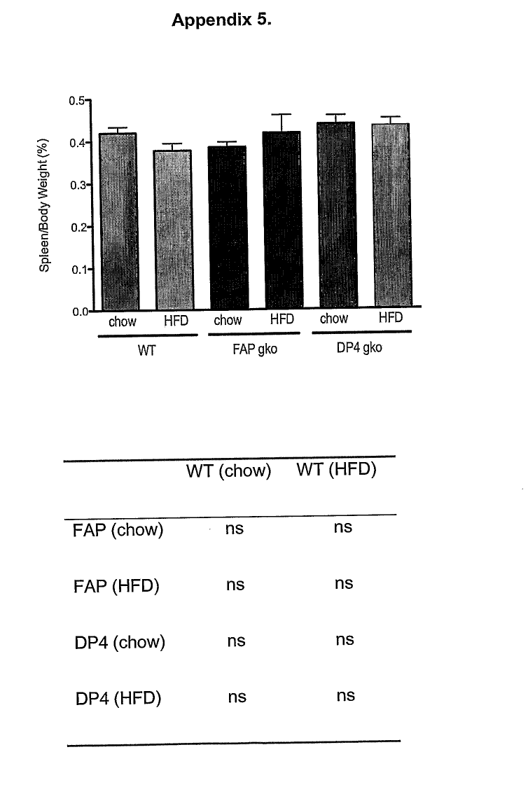

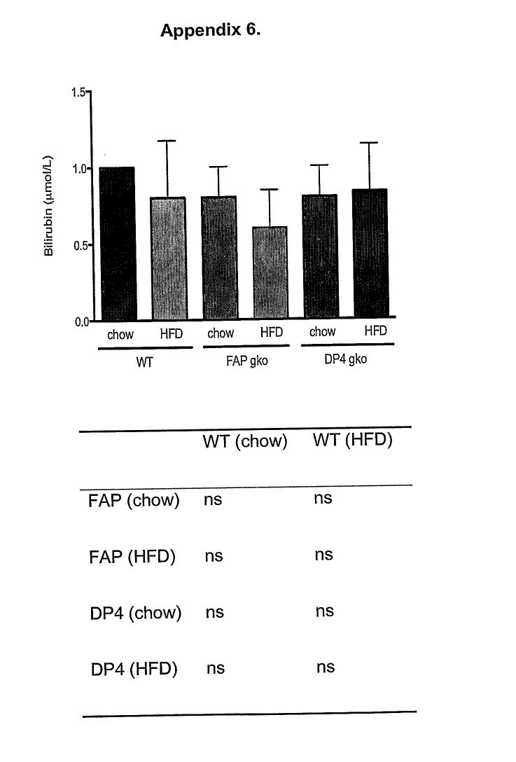

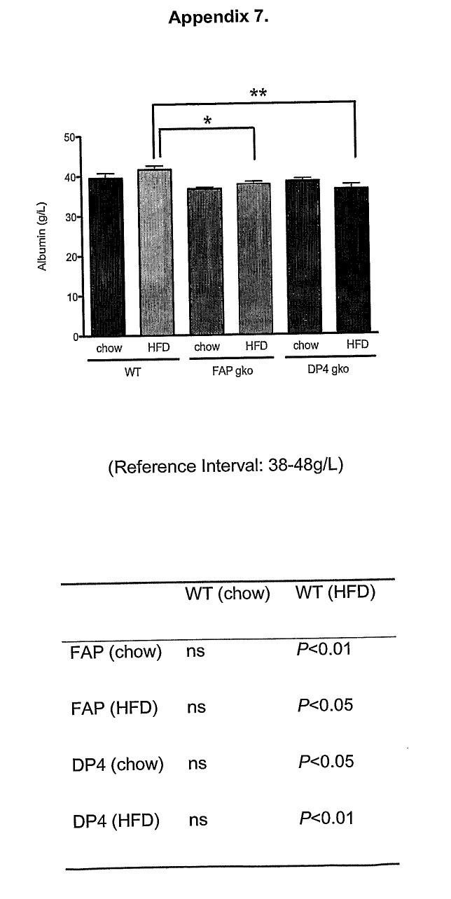

Realtime RT-PCR analysis was used to analyse mRNA expression of stearoyl-CoA desaturase 1 (SCD) and acetyl-Coenzyme A carboxylase (ACC) in mouse livers (FIG. 10). FAP gko mice on chow exhibited significantly lower mRNA expression levels of both SCD and ACC, suggesting less lipid synthesis in FAP gko mice.

### Discussion

These data show that lack of FAP increases glucose tolerance and confers protection against HFD consequences including elevated blood glucose and impaired glucose tolerance. In addition, lacking FAP was associated with less SCD and ACC, which probably caused less liver lipid synthesis.

### Example 3

### Aim

To demonstrate FAP expression in normal (non-fibrotic) tissue.

### Materials & Methods

Animals: Baboon (Papio hamadryas) tissue samples were obtained from the primate colony maintained by the Royal Prince Alfred Hospital under NHMRC and hospital regulations and approvals.

FAP Immunoblot Analysis method.

Cells and frozen tissue samples were lysed in Triton-based (20 mM Tris-HCl pH 7.6, 10 mM MgCl₂, 2 mM EDTA, 10% glycerol, 1% Triton-114, protease inhibitor cocktail (Roche)). Gels used were 3-8% Tris-Acetate SDS-PAGE, 4-12% Bis-Tris SDS-PAGE (Invitrogen). PVDF blots were probed with anti-FAP monoclonal antibody [MAb] F19 (diluted 1:3) or 1E5 (1 ug/ml) as described (7, 13,15). Anti-GAPDH MAb MCA-1DC (EnCor Biotechnology Inc) stained GAPDH as the loading control. Recombinant soluble human FAP has been described (15).

### Purified Human FAP.

Baculovirus—expressed soluble human FAP (residues 39-760) polyhistidine-tagged at the C terminus was purified by metal affinity chromatography and visualised on silver-stained 3-8% SDS-PAGE immunoblot with MAb 1E5 (Abnova, Taipei, Taiwan, Catalogue No. H00002191-M01) and with MAb F19 (ATCC, Manassas, Va., Hybridoma catalogue No. CRL-2733) (C) followed by horseradish peroxidase conjugated rabbit anti-mouse IgG (DAKO, Santa Barbara, Calif., diluted 1:3000)

### Results & Discussion

MAbs F19 and 1E5 both bind to recombinant human FAP in immunoblots, but to different epitopes (13). We hypothesised that the 1 E5 epitope may be better preserved than the F19 epitope in analytical methods of detecting FAP. MAb 1 E5 readily detected a protein having similar molecular weight characteristics to FAP in adrenal, kidney, small intestine and seminal gland. The baboon and human genomes are 96% identical and the physiologies are very similar, so it is very likely that FAP protein is also present at significant levels in human tissues. A further indication that FAP protein is more prevalent than previously thought is that FAP has been purified from bovine serum (19). This method of FAP detection similarly did not rely upon MAb F19.

Incretins are produced by the small intestine and are present in serum, so the presence of FAP in serum and small intestine shows potential for FAP to contact and degrade incretins such as GLP-1.

### Example 4

Lowering Elevated Blood Glucose with FAP Specific Inhibitors

### Aim

To determine efficacy and FAP dependence of FAP specific inhibitors for lowering elevated blood glucose levels in vivo

### Methods

### Mice

Female mice of C57BL/6 (WT), and FAP gko on C57BL/6 genetic background are housed in Centenary Institute under University of Sydney animal ethics committee approvals.

### FAP Specific Inhibitor Titration

Mice aged 8-12 weeks (n=6) are treated with FAP specific inhibitor. FAP specific inhibitor as described herein is titred in vivo to determine a dosage that lowers the level of FAP dipeptidyl peptidase activity detected in plasma by at least 90%. Doses of FAP specific inhibitor of 0.1 to 50 mg/Kg/day are administered to mice i.p. [intraperitoneally] daily or twice—daily for 5 to 7 days. Inhibitor diluent as negative control. Plasma FAP activity is measured in this assay: Enzyme assay at 37 degrees C. using H-AlaPro-pNA as the substrate and, 15-20 minutes before adding substrate, adding a selective DPP4 inhibitor such as sitagliptin at 10 micromolar to inhibit DPP4 and 2 mM NEM [N-ethylmaleimide] to inhibit DP8 and DP9. Buffer is 50 mM Tris 10 mM EDTA pH 7.9-8.0.

Measure absorbance at 405nm and at 572 nm at 5 min and 15 min after adding substrate: enzyme activity in OD/min is (A405-A572)/10. Example 3 shows that kidney is a rich source of FAP, so at autopsy kidney FAP is assayed to further verify FAP enzyme activity ablation.

In the FAP enzyme assay an alternative method is to use H-AlaPro-AFC as the substrate and measure fluorescence [excitation filter 485 nm, emission filter 510 nm] at the 5 minute, 15 minute and 25 minute time points.

### Study Groups

Group 1—WT mice receiving FAP specific inhibitor
Group 2—WT mice receiving inhibitor diluent only.
Group 3—FAP gko receiving FAP specific inhibitor
Group 4—FAP gko receiving inhibitor diluent only.

### Intraperitoneal Glucose Tolerance Test (IPGTT)

Mice are treated with FAP specific inhibitor for 5 to 7 days, as determined and described above, and plasma FAP inhibition confirmed. Then these mice are fasted for 5 hours before receiving intraperitoneal administration of 4 g of D-glucose per kg body weight in saline (0.9% NaCl). Blood samples of conscious mice are collected from the tail vein at 0, 30, 60, 120 and 180 min. Blood glucose concentration is determined on Accucheck Performa (Roche Diagnostic). Groups 1 and 3 are given FAP specific inhibitor.

### Oral Glucose Tolerance Test (OGTT)

Mice are treated with FAP specific inhibitor at the amount determined and described above, and plasma FAP inhibition confirmed. Then these mice (n=6 per group) continue to be treated with inhibitor while being fed ad libitum for 8 weeks either chow and water or a high fat diet (HFD) (23% fat, Cat No. SF03-030, Specialty Feeds, Perth, West Australia) and with water supplemented with 5% fructose (Sigma, USA). The mice are then fasted for 5 hours before oral administration of 4 g of D-glucose per kg body weight in saline (0.9% NaCl). Blood samples are collected and assayed as described above. Groups 1 and 3 are given FAP specific inhibitor.

Additional measurements in HFD fed mice; Standard assay methods can be used for these parameters. These include plasma insulin, leptin, adiponectin, lipids, ALT and AST. Also liver glucokinase and lipid content. Liver mRNA levels of lipid metabolism genes SCD and ACC, adiposity [adipose tissue weight/body weight], food intake, total and % fecal fat content are measured. Plasma concentration of active GLP-1 is measured [kit from Linco].

### Statistical Analysis

Area Under the Curve [AUC] of plot of glucose concentration versus time is calculated for each group. The glucose concentration and AUC data are presented as mean±SEM. Student's T test is used to compare between groups. P values<0.05 are considered significant.

### Discussion

FAP specific inhibitor—treated WT mice have a smaller AUC than diluent—treated WT mice where a FAP specific inhibitor increases glucose tolerance. The glucose response does not differ between FAP specific inhibitor—treatment and diluent—treatment in FAP gko mice where the increased glucose tolerance from a FAP specific inhibitor is due to its inhibition of FAP. These findings demonstrate the potential for FAP specific inhibitor to treat impaired glucose tolerance and type 2 diabetes. The additional measurements demonstrated protection from obesity, and lowered insulin sensitivity, hepatocyte injury and hepatic steatosis.

### REFERENCES

1. Charbonnel B, Karasik A, Liu J, Wu M, Meininger G. Efficacy and safety of the dipeptidyl peptidase-4 inhibitor sitagliptin added to ongoing metformin therapy in patients with type 2 diabetes inadequately controlled with metformin alone. Diabetes Care 2006 Dec;29(12):2638-2643.
2. Conarello S, Li Z, Ronan J, Roy R, Zhu L, Jiang G, et al. Mice lacking dipeptidyl peptidase IV are protected against obesity and insulin resistance. Proceedings of the National Academy of Sciences United States of America 2003 MAY 27;100(11):6825-6830.
3. Ding X, Saxena N K, Lin S, Gupta N A, Anania F A. Exendin-4, a glucagon-like protein-1 (GLP-1) receptor agonist, reverses hepatic steatosis in ob/ob mice. Hepatology 2006;43(1):173-181.
4. Eng J, Kleinman W A, Singh L, Singh G, Raufman J R Isolation and characterization of exendin-4, an exendin-3 analogue, from Heloderma suspect=venom. Further evidence for an exendin receptor on dispersed acini from guinea pig pancreas. The Journal of biological chemistry 1992 Apr 15;267(11):7402-7405.
5. Gorrell M D. Dipeptidyl peptidase IV and related enzymes in cell biology and liver disorders. Clinical Science 2005 01/04/2005108(4):277-292.
6. Gorrell M D, Yu D M T. Diverse functions in a conserved structure: The dipeptidyl peptidase IV gene family. In: Robinson J W, editor. Trends in Protein Research. New York: Nova Science Publishers, Inc.; 2005. p. 1-78.
7. Levy M T, McCaughan G W, Abbott C A, Park J E, Cunningham A M, Muller E, et al. Fibroblast activation protein: a cell surface dipeptidyl peptidase and gelatinase expressed by stellate cells at the tissue remodelling interface in human cirrhosis. Hepatology 1999 Jun;29(6):1768-1778.
8. Levy M T, McCaughan G W, Marinos G, Gorrell M D. Intrahepatic expression of the hepatic stellate cell marker fibroblast activation protein correlates with the degree of fibrosis in hepatitis C virus infection. Liver International 2002 Apr;22(2):93-101.
9. Marguet D, Baggio L, Kobayashi T, Bernard A M, Pierres M, Nielsen P F, et al. Enhanced insulin secretion and improved glucose tolerance in mice lacking CD26. Proceedings of the National Academy of Sciences of the United States of America 2000 Jun 6;97(12):6874-6879.
10. Park J E, Lenter M C, Zimmermann R N, Garin-Chesa P, Old L J, Rettig W J. Fibroblast activation protein: A dual-specificity serine protease expressed in reactive human tumour stromal fibroblasts. Journal of Biological Chemistry 1999;274:36505-36512.
11. Pospisilik J A, Stafford S G, Demuth H U, Brownsey R, Parkhouse W, Finegood D T, et al. Long-term treatment with the dipeptidyl peptidase IV inhibitor P32/98 causes sustained improvements in glucose tolerance, insulin sensitivity, hyperinsulinemia, and beta-cell glucose responsiveness in VDF (fa/fa) Zucker rats. Diabetes 2002;51(4):943-950.
12. Rosenstock J, Brazg R, Andryuk P J, Lu K, Stein P, Sitagliptin Study G. Efficacy and safety of the dipeptidyl peptidase-4 inhibitor sitagliptin added to ongoing pioglitazone therapy in patients with type 2 diabetes: a 24-week, multicenter, randomized, double-blind, placebo-controlled, parallel-group study. Clinical Therapeutics 2006;28(10):1556-1568.
13. Wang X M, Yao T-W, Nadvi N A, Osborne B, McCaughan G W, Gorrell M D. Fibroblast activation protein and chronic liver disease. Frontiers in Bioscience 2008 Jan. 1, 2008;13:3168-3180.
14. Andrikopoulos S, Blair A R, Deluca N, Fam B C, Proietto J. Evaluating the glucose tolerance test in mice. American journal of physiology 2008 Dec;295(6):E1323-1332.
15. Reversible inactivation of human dipeptidyl peptidases 8 and 9 by oxidation. The Open Enzyme Inhibition Journal 1: 52-61.Park J, Knott H M, Nadvi N A, Collyer C A, Wang X M, Church W B, Gorrell M D (2008)
16. Seth, D, P J Hogg, M D Gorrell, G W McCaughan, P S Haber 2008 Direct effects of alcohol on hepatic fibrinolytic balance: implications for alcoholic liver disease. J Hepatol, 48: 614-27
17. Aggarwal S, Brennen W N, Kole T P, Schneider E, Topaloglu O, Yates M, Cotter R J, Denmeade SR (2008). Fibroblast activation protein peptide substrates identified from human collagen I derived gelatin cleavage sites. Biochemistry 47(3): 1076-1086.
18. Lee K N, Jackson K W, Christiansen V J, Lee C S, Chun J G, McKee P A (2006). Antiplasmin-cleaving enzyme is a soluble form of fibroblast activation protein. Blood 107(4): 1397-1404.
19. Collins P J, McMahon G, O'Brien P, O'Connor B (2004). Purification, identification and characterisation of seprase from bovine serum. International Journal of Biochemistry and Cell Biology 36: 2320-2333.

1. Use of a FAP selective inhibitor for one or more of:
controlling blood glucose level;
increasing insulin secretion;
decreasing glucagon secretion;
increasing β cell mass and insulin gene expression;
inhibiting acid secretion and gastric emptying in the stomach;
decreasing food intake by increasing satiety.

2. A method of preventing or treating one or more of the following diseases or conditions:
impaired glucose tolerance;
impaired fasting glucose;
insulin resistance;
new onset diabetes mellitus;
metabolic syndrome;
diabetes mellitus;
hepatic steatosis;
the method including providing a FAP selective inhibitor to an individual having one or more the above diseases or conditions, or to an individual susceptible for one or more of the above diseases or conditions.

3. The method of claim 2, wherein the individual susceptible for one or more of the above diseases or conditions has
a pre-diabetic state.

4. A use of a FAP selective inhibitor in the manufacture of a medicament for preventing or treating one or more of the diseases or conditions according to claim 2.

5. A method or use according to claim 1 wherein said FAP selective inhibitor does not substantially inhibit the dipeptidyl peptidase activity of DPIV.

6. The method or use according to claim 5 wherein said FAP selective inhibitor does not substantially inhibit the dipeptidyl peptidase activity of DP8, DP9 or PEP.

7. The method or use according to claim 1 wherein the inhibitor is selected from the group consisting of
(2S)-1-((2 S)-2-(2-Methoxybenzoylamino)-3-methylpentanoyl) pyrrolidine-2-carbonitrile;
(2S)-1-((2 S)-2-(Ethylcarbamoylamino)-3-methylpentanoyl) pyrrolidine-2-carbonitrile;
(2S)-1-((2 S)-2-(3-Methoxybenzoylamino)-3-methylpentanoyl) pyrrolidine-2-carbonitrile;
(2S)-1-(2-(N-Benzyl-N-(2-methoxybenzoyl) amino)acetyl)pyrrolidine-2-carbonitrile;
(2S)-1-(2-(2-Methoxybenzoylamino)-2-methyl propanoyl)pyrrolidine-2-carbonitrile;
(2S)-1-((2S)-2-(Ethylcarbamoylamino)-3-methylbutanoyl)pyrrolidine-2-carbonitrile;
(2S)-1-a2S)-2-(Isopropylcarbamoylamino)propanoyl)-pyrrolidine-2-carbonitrile;
(2S)-1-((2S)-2-(Ethylcarbamoyl)octahydroindole-2-carbonyl)pyrrolidine-2-carbonitrile;
(2S)-1-((2S)-2-(N-Ethylcarbamoyl-N-methylamino)-4-methylpentanoyl) pyrrolidine-2-carbonitrile;
(2S)-1-((2S)-2-(Benzylcarbamoylamino)-3, 3-dimethylbutanoyl)pyrrolidine-2-carbonitrile;
(2S)-1-((2S)-2-(Isopropylcarbamoylamino)-3-phenylpropanoyl)pyrrolidine-2-carbonitrile;
(2S)-1-((2S)-2-(Cyclohexylcarbamoylamino)-4-methylpentanoyl)pyrrolidine-2-carbonitrile; and
(2S)-1-((2S)-2-(Ethylcarbamoylamino)-3-methylpentanoyl)-3,4-dehydropyrrolidine-2-carbonitrile.

8. The method or use according to claim 1 wherein the inhibitor is selected from the group consisting of
(2S)-2-Formyl-1-((2S)-2-(2-methoxybenzoylamino)-3-methylbutanoyl)pyrrolidine;
(2S)-2-Formyl-1-((2S)-1-(3-methoxybenzoyl)pyrrolidine-2-carbonyl)pyrrolidine;
(2S)-2-Formyl-1-((2S)-1-(ethylcarbamoyl)octahydroindole-2-carbonyl)pyrrolidine;
(2S)-2-Formyl-1-((2S)-2-(2-methoxybenzoyl)-octahydroindole-2-carbonyl) pyrrolidine; and
(2S)-2-Formyl-1-(2S)-2-(2-methoxybenzoylamino)-3-phenylpropanoyl)pyrrolidine.

9. The method or use according to claim 1 wherein the inhibitor is a compound represented by Formula A: 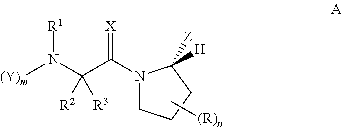
or a pharmaceutically acceptable salt thereof, wherein, independently for each occurrence:
X represents O, S, or NR;
Y represents H, naturally occurring L-amino acid residue, naturally occurring D-amino acid residue, or Λ/-terminal protecting group;
Z represents —CO₂R , —SO₃H, —SO₂NH₂, —B(OH)₂, —PO₃H₂, or 5-tetrazolyl;
R represents independently for each occurrence H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, or arylsulfonyl;
R¹ represents H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, or arylsulfonyl;
R² represents H, a side chain of a naturally occurring amino acid, or a side chain of a non-naturally occurring amino acid;
R³ represents H, a side chain of a naturally occurring amino acid, or a side chain of a non-naturally occurring amino acid;
R¹ and R² may be taken together to form an 3-8 member ring that may be optionally substituted;
R¹ represents, independently for each occurrence, H, alkyl, alkenyl, alkynyl, aryl, heteroaryl, aralkyl, or heteroaralkyl;
m is an integer in the range 1 to about 10; and
n is an integer in the range 0 to 6.

10. The method or use according to claim 1 wherein the inhibitor is a compound represented by Formula B: 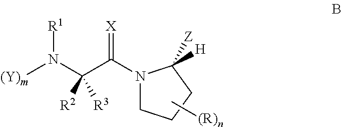
or a pharmaceutically acceptable salt thereof, wherein, independently for each occurrence:
X represents O, S, or NR;
Y represents H, naturally occurring L-amino acid residue, naturally occurring D-amino acid residue, or Λ/-terminal protecting group;
Z represents —CO₂R¹, —SO₃H, —SO₂NH₂, —B(OH)₂, —PO₃H₂, or 5-tetrazolyl;
R represents independently for each occurrence H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy)carbonyl, (arylalkyloxy)carbonyl, (alkylamino)carbonyl, (arylamino)carbonyl, (arylalkylamino)carbonyl, alkylsulfonyl, or arylsulfonyl;
R¹ represents H, substituted or unsubstituted alkyl, cycloalkyl, alkenyl, aryl, heteroaryl, arylalkyl, cyano, halogen, hydroxyl, alkoxyl, aryloxy, arylalkyloxy, amino, alkylamino, arylamino, arylalkylamino, sulfhydryl, alkylthio, arylthio, arylalkylthio, nitro, azido, alkylseleno, formyl, acyl, carboxy, silyl, silyloxy, (alkyloxy)carbonyl, (aryloxy) carbonyl, (arylalkyloxy)carbonyl, (alkylamino) carbonyl, (arylamino)carbonyl, (arylalkylamino)carbonyl, alkylsulfonyl, or arylsulfonyl;
R² represents H or a side chain of a naturally occurring amino acid;
R³ represents H or a side chain of a non-naturally occurring amino acid;
R¹ and R² may be taken together to form an 3-8 member ring that may be optionally substituted;
R¹ represents, independently for each occurrence, H, alkyl, alkenyl, alkynyl, aryl, heteroaryl, aralkyl, or heteroaralkyl;
m is an integer in the range 1 to about 10; and
n is an integer in the range 0 to 6.

11. A kit for use in a method or use according to claim 1, the kit including:
a container holding a FAP selective inhibitor;
a label or package insert with instructions for use.
